(19) United States
(12) Patent Application Publication
(10) Pub. No.: US 2022/0298234 A1
(43) Pub. Date: Sep. 22, 2022

(54) MACROPHAGE DIVERSITY IN REGENERATIVE, FIBROTIC BIOMATERIAL ENVIRONMENTS

(71) Applicant: The Johns Hopkins University, Baltimore, MD (US)

(72) Inventors: Jennifer Elisseeff, Baltimore, MD (US); Christopher Cherry, Baltimore, MD (US); Sven Daniel Sommerfeld, Baltimore, MD (US)

(21) Appl. No.: 17/611,028

(22) PCT Filed: May 13, 2020
(86) PCT No.: PCT/US2020/032714

§ 371 Date: Nov. 12, 2021

Related U.S. Application Data
(60) Provisional application No. 62/846,769, filed on May 13, 2019.

(51) Int. Cl. C07K 16/24; A61L 27/54; A61P 37/06; A61K 38/20

(52) U.S. Cl. CPC C07K 16/244; A61L 27/54; A61P 37/06; A61K 38/20; A61L 2300/432

(57) ABSTRACT

The present invention provides methods for identification of distinct macrophage subsets which demonstrate previously unrecognized myeloid macrophage phenotypes involved in different tissue responses and provide new methods for therapeutic modulation of certain pathologic tissue states and tissue repair.

### REFERENCE TO RELATED APPLICATIONS

This application claims the benefit of U.S. Provisional Patent Application No. 62/846,769, filed on May 13, 2019, which is hereby incorporated by reference for all purposes as if fully set forth herein.

### BACKGROUND OF THE INVENTION

Macrophages are immune cells of myeloid lineage that maintain tissue homeostasis and participate in host defense. They remove cell debris, recycle, and clear apoptotic cells during tissue homeostasis and remodeling. As members of the innate immune system, macrophages sense and respond to infection, cancer, and tissue damage by cytokine and growth factor secretion and phagocytic activity. They are required for tissue regeneration and repair in addition to host defense. Through their cytokine profile and antigen presentation capacity, macrophages can engage and influence the adaptive immune system. For example, tumor-associated macrophages are a component of the tumor immune microenvironment where depending on their phenotype, they promote an immunosuppressive program that supports tumor growth or alternatively prime anti-tumor T cells to promote tumor regression (1, 2). Similarly, the wound associated macrophage phenotype can determine repair and fibrosis outcomes depending on T cell effector state (3-5).

Macrophages execute varied programs through their functional diversity and plasticity. They are highly specialized depending on tissue environment; i.e., alveolar macrophages in the lung (6), Kupffer cells in the liver (7), and microglial cells in the brain (8). Perturbations to the environment, particularly associated with certain disease states, alter their phenotype and function from normal tissue imprinting. The local tissue environment can influence macrophage phenotype beyond their developmental lineage. For example, environmental factors regulate macrophage epigenetics to control progenitor differentiation and reprogramming of already differentiated macrophages (9). In the tissue response to trauma and foreign bodies, macrophages play successive roles from initial inflammation to innate defense and resolution, resulting in tissue repair or chronic inflammation and fibrosis depending on environmental signals (10).

Macrophage functional heterogeneity is key to their ability to respond to diverse environments and cues. Yet this complexity in activity and gene expression is not reflected by current phenotyping and nomenclature dogma. Early attempts to classify macrophages resulted in the M1/M2 nomenclature to define the pro-inflammatory, interferon gamma (IFNγ) activated macrophage (M1) versus the “alternatively IL(Interleukin)-4 activated” macrophage (M2) (11, 12). Following this, a spectrum model of macrophage activation was proposed (13). In recent years, a common framework for macrophage activation has been suggested that considers a set of surface and genetic markers including CD206 as an “M2 marker” and CD86 as an “M1 marker” (14). Recent technological advances, however, including single cell RNA sequencing (scRNAseq) and mass cytometry suggest that archetypal in vitro phenotypes rarely overlap with those found in physiological conditions (15, 16). These approaches open the door to the refined characterization of macrophage populations that reflects the complexity of their phenotype and function in normal and pathological environments.

Biomaterials generate tissue microenvironments that can reproducibly induce specific macrophage phenotypes. Biological scaffolds, derived from the extracellular matrix (ECM) of tissues, promote a pro-regenerative tissue microenvironment that induces tissue repair through increased expression of IL-4. The repair capacity of these materials correlates with a T_{H}2 T cell response that directs polarization to a traditionally-defined M2 macrophage in combination with a reduction of CD86 expression (3, 4). Synthetic materials induce a foreign body response (FBR) that has been associated with inflammatory M1-type macrophages and development of a fibrotic capsule (17, 18). While the M1-type inflammatory macrophage has been conventionally associated with a T_{H}1 response, we have found that the FBR and the associated macrophage phenotype occurs in a type 17 immune environment that includes IL-17 production by innate lymphocytes, γδ T cells, and T_{H}17 T cells. IL-17 signaling is required for the fibrosis associated with the FBR, though the level of IL-17 expression may vary depending on the chemical and physical properties of the materials (19). Synthetic and biological materials, therefore, serve as a model for Type 2 and Type 17 tissue immune microenvironments where the associated macrophage phenotype can be studied.

As such, there remains a critical need for new systems of macrophage classification that correlates with the complexity of their phenotype and function in normal and pathological environments.

### SUMMARY OF THE INVENTION

In accordance with multiple embodiments, the present inventors use scRNAseq to characterize macrophages isolated from a murine model of tissue repair versus fibrosis. Unbiased clustering algorithms revealed diverse and novel populations of macrophages. The inventors identified phenotypic properties of the macrophage clusters that were unique to a regenerative or fibrotic biomaterial tissue environment. The inventors then identified surface markers that defined the macrophages clusters and validated their ability to separate macrophage subsets experimentally by flow cytometry and immunofluorescence. Finally, the broader relevance of the macrophage subsets in murine and human tissue pathologies was validated.

In accordance with an embodiment, the present invention provides a method for reducing or treating the progression of a fibrosis associated disease or condition in a subject in need thereof, comprising reducing or inhibiting a cell or population of cells expressing the F1 and/or F2 macrophage subtype in the subject.

In accordance with another embodiment, the present invention provides a method for reducing or inhibiting a cell or population of cells expressing the F1 and/or F2 macrophage subtype in a subject in need thereof, comprising administering to the subject an effective amount of an IL-17 and/or IL36γ inhibitor.

In accordance with an embodiment, the present invention provides a method for reducing or treating the progression of a fibrosis associated disease or condition in a subject in need thereof, comprising reducing or inhibiting a cell or population of cells expressing the F1 macrophage subtype in the subject by inhibition of the transcription or expression of the IL-18 gene in the cell or population of cells in the subject.

In accordance with an embodiment, the present invention provides a method for reducing or treating the progression of a fibrosis associated disease or condition in a subject in need thereof, comprising administering to the subject an effective amount of an IL-18 inhibitor.

In accordance with an embodiment, the present invention provides a method for reducing or treating the progression of a fibrosis associated disease or condition in a subject in need thereof, comprising reducing or inhibiting a cell or population of cells expressing the F2 macrophage subtype in the subject by inhibition of the transcription or expression of one or more of the genes selected from the group consisting of: Il36 gamma, Il17 receptor A, triggering receptor expressed on myeloid cells 1 (Trem-1), aspartic peptidase retroviral like 1 (Asprv1), Toll-like receptor 2 (Tlr2), secretory leukocyte peptidase inhibitor (Slpi), and histidine decarboxylase (Hdc), in the cell or population of cells in the subject.

In accordance with an embodiment, the present invention provides a method for reducing or treating the progression of a fibrosis associated disease or condition in a subject in need thereof, comprising administering to the subject an effective amount of an inhibitor of one or more of the proteins selected from the group consisting of: Il36 gamma, Il17 receptor A, triggering receptor expressed on myeloid cells 1 (Trem-1), aspartic peptidase retroviral like 1 (Asprv1), Toll-like receptor 2 (Tlr2), secretory leukocyte peptidase inhibitor (Slpi), and histidine decarboxylase (Hdc). In some embodiments the inhibitors can also target one or more additional proteins selected from the group consisting of: transmembrane protein 1, lipocalin 2, leucine rich alpha-2-glycoprotein 1, and C—X—C motif chemokine receptor 2.

In accordance with an embodiment, the present invention provides a method for reducing or treating the progression of a fibrosis associated disease in a subject in need thereof comprising administering to the subject an effective amount of an IL-17 and/or IL36γ inhibitor.

In accordance with an embodiment, the present invention provides a method for reducing or treating the progression of fibrosis in a subject having an autoimmune disease comprising administering to the subject an effective amount of an IL-17 and/or IL36γ inhibitor.

In accordance with an embodiment, the present invention provides a method for improving regenerative healing in a wound of a subject in need thereof, comprising increasing a cell or population of cells expressing the R1 and/or R2 macrophage subtype in the subject.

In accordance with an embodiment, the present invention provides a method for improving regenerative healing in a wound of a subject in need thereof, comprising increasing a cell or population of cells expressing the R2 macrophage subtype by inhibition of proteins associated with the R1 subtype, including granzyme A (CTLA 3, Gzma), CD52 CAMPATH 1-Antigen (which is inhibited by Alemtuzumab, for example), lipoprotein lipase, CD209, and C—C motif chemokine receptor 2, for example.

In accordance with an embodiment, the present invention provides a method for improving regenerative healing in a wound of a subject in need thereof, comprising any of the methods described herein and further comprising administering to the subject one or more cytokines secreted by a cell or population of cells expressing the R2 macrophage subtype including, for example, C—C motif chemokine ligand 8 (CCl8) C—C motif chemokine ligand 24 (CCl24).

In accordance with an embodiment, the present invention provides a method for improving regenerative healing in a wound of a subject in need thereof, by increasing a cell or population of cells expressing the R1 and/or R2 macrophage subtype comprising increasing the transcription or expression of the CD301 gene in the cell or population of macrophage cells in a subject.

In accordance with an embodiment, the present invention provides a method for improving regenerative healing in a wound of a subject in need thereof, by increasing a cell or population of cells expressing the R1 and/or R2 macrophage subtype comprising increasing the transcription or expression of the monoglyceride lipase (MGL) gene in the cell or population of macrophage cells in a subject.

In accordance with an embodiment, the present invention provides a method for reducing or treating the progression of a fibrosis at the site of a surgical procedure in a subject comprising administering to the site of the subject an effective amount of an IL-17 and/or IL36γ inhibitor.

In accordance with an embodiment, the present invention provides a method for reducing or treating the progression of a fibrosis at the site of a surgical implant in a subject comprising coating the implant with an effective amount of an IL-17 and/or IL36γ inhibitor prior to implantation in the subject.

In accordance with an embodiment, the present invention provides a method for identification of regenerative associated macrophages in a heterogeneous cellular sample, the method comprising: contacting the heterogeneous cellular sample comprising regenerative associated macrophages, fibrotic associated macrophages, and other macrophages with a CD301 specific binding member; distinguishing the regenerative associated macrophages based on whether the CD301 cell surface marker specific binding member binds to a cell surface marker on macrophages of the sample.

In accordance with an embodiment, the present invention provides a method for identification of a subpopulation of regenerative associated macrophages known as R1 macrophages in a heterogeneous cellular sample, the method comprising: contacting a sample of the regenerative associated macrophages that were distinguished with a CD301 specific binding member binding to the macrophages above with a CD9 and MHCII specific binding member; distinguishing the R1 macrophages based on whether the CD9 and MHCII surface marker specific binding members both bind to the cell surface markers on macrophages of the sample.

In accordance with an embodiment, the present invention provides a method for identification of a subpopulation of regenerative associated macrophages known as R2 macrophages in a heterogeneous cellular sample, the method comprising: contacting a sample of the regenerative associated macrophages that were distinguished with a CD301 specific binding member binding to the macrophages above with a CD9 and MHCII specific binding member; distinguishing the R2 macrophages based on whether neither of the CD9 and MHCII surface marker specific binding members bind to the cell surface markers on macrophages of the sample.

In accordance with an embodiment, the present invention provides a method for identification of fibrotic associated macrophages in a heterogeneous cellular sample, the method comprising: contacting the heterogeneous cellular sample comprising regenerative associated macrophages, fibrotic associated macrophages, and other macrophages with a CD301 specific binding member; distinguishing the fibrotic associated macrophages based on whether the CD301 cell surface marker specific binding member does not bind to a cell surface marker on macrophages of the sample.

In accordance with an embodiment, the present invention provides a method for identification of a subpopulation of fibrotic associated macrophages known as F1 macrophages in a heterogeneous cellular sample, the method comprising: contacting a sample of the fibrotic associated macrophages that were distinguished with a CD301 specific binding member which did not bind to the macrophages in the previous paragraph with a CD9 and MHCII specific binding member; distinguishing the F1 macrophages based on whether the CD9 surface marker specific binding member does not bind to the cell surface markers and the MHCII specific binding member does bind to the cell surface markers on macrophages of the sample.

In accordance with an embodiment, the present invention provides a method for identification of a subpopulation of fibrotic associated macrophages known as F2 macrophages in a heterogeneous cellular sample, the method comprising: contacting a sample of the fibrotic associated macrophages that were distinguished with a CD301 specific binding member which did not bind to the macrophages in the previous paragraph with a CD9 and MHCII specific binding member; distinguishing the F2 macrophages based on whether the CD9 surface marker specific binding member binds to the cell surface markers and the MHCII specific binding member does not bind to the cell surface markers on macrophages of the sample.

In accordance with an embodiment, the present invention provides a method for monitoring the progression of a fibrosis associated disease in a subject in need thereof comprising measuring a first subpopulation of F1 and/or F2 macrophages in a heterogeneous cellular sample from the subject, measuring one or more subsequent subpopulation samples of F1 and/or F2 macrophages.

### BRIEF DESCRIPTION OF THE DRAWINGS

FIGS. 1A-1E. Single cell characterization of macrophages in fibrotic and regenerative microenvironments. (1A) Experimental overview. A virtual aggregate of macrophages in fibrosis and regeneration generated from single cell RNA-seq after sorting of F4/80^{hi+}CD64⁺ cells isolated from murine volumetric muscle injuries at 1 week, treatment with biomaterials UBM (regenerative), synthetic (fibrotic), or saline (control). (1B) Heat map of differentially expressed genes. Up to 200 cells per cluster are shown, ordered by cluster, with the top 10 differentially expressed genes. Functionally relevant genes from terminal clusters are annotated. (1C) Dimensional reduction projection of cells onto two dimensions using uniform manifold projection approximation (UMAP). Cells are colored by experimental biomaterial condition (top) and computationally determined cluster (bottom). (1D) Summary of cluster differentiation trajectories, markers, and biological functions generated by bioinformatics analysis. (1E) A flow cytometry strategy informed by in silico determined markers including CD9, CD301b and MHCII differentiating in vivo macrophage subsets from UBM, synthetic. Subsets are colored equivalent to in silico clusters, back gated into tSNE projection.

FIGS. 2A-2G. ScRNAseq reveals surface markers that discriminate diverse regenerative and fibrotic macrophage clusters. (2A) Histogram of CD86 and CD206 expression of bulk macrophages from regenerative, fibrotic, and saline microenvironments (green, red, blue) by flow cytometry. (2B) Feature plots of in silico Cd86 (bottom) and Cd206 (Mrc1, top) expression superimposed on UMAP plots of cells from scRNAseq. Circled borders mark regions enriched for cells from the regenerative or fibrotic experimental condition. (2C) Cluster expression of canonical M1 (red) and M2 (green) markers. Expression levels are given as cluster averages normalized to the maximum value per gene. (2D) Gene expression (UMI count) of M1 and M2 markers per single cell. Cells are ordered and colored by condition (regenerative as green, saline as blue, and fibrotic as red). (2E) Violin plots of cluster in silico gene expression for surface markers. Surface markers were identified by differential expression analysis. (2F) Flow cytometry gating strategy using CD9 and CD301b differentiates fibrotic and regenerative macrophage subsets in vivo. (2G) Mean fluorescence values indicate subset specific expression of activation markers CD86 and CD206 (n=4 biologically independent, **p<0.005, ****p<0.0001).

FIGS. 3A-3J. In silico RACs reveals distinct in vivo regenerative macrophage phenotypes including muted-inflammatory R1 and phagocytotic R2. (3A) Predicted lineage schematic of RACs from Slingshot pseudotime analysis. In silico predicted surface markers are shown. (3B) Slingshot pseudotime trajectory of RACs shown on a principal component plot (PC1 vs. PC2). Cells are colored by cluster. (3C) Heat map of top 20 differentially expressed genes in a comparison of R1 and R2. (3D) Violin plots for differentially expressed genes comparing R1 and R2. (3E) Gene set enrichment comparing R1 and R2. Plots with higher peaks (red) indicates enrichment of gene sets in R2 while plots with negative peaks (blue) indicate enrichment of gene sets in R1. (3F) Gene network plots of R1 and R2 generated. Nodes represent genes with connections generated by STRING metadata analysis. Sets of genes associated with specific functions are annotated. (3G) Flow cytometry gating scheme validates in vivo protein marker combination for R1 and R2. Macrophages defined as F4/80^{hi+} from live, CD45⁺. (3H) 1-6 weeks' time course of R1 and R2 subsets in UBM, PCL, and saline microenvironments (n=4 biologically independent). Two-way analysis of variance with subsequent multiple testing p values are presented. (****p<0.0001). (3I) Phagocytosis of flash red fluorescent microbeads by ex vivo cultured, sorted R1 and R2 macrophages, arrows indicate poly(styrene) bead locations (scale bar=50 μm).

FIGS. 4A-4D. Cluster R3 expresses tissue specific genes. (4A) UMI counts for genes overexpressed in R3. Genes shown here are associated with skeletal muscle function. (4B) Violin plots of genes associated with endocytosis and lysosome activity. (4C) Network analysis of F2 genes. Nodes are genes with edges connecting nodes representing connections in databases or literature. Modules of genes with associated functions are annotated. (4D) Gene set enrichment on differential expression comparison of R3 to all other macrophages. Running enrichment plots (top) show high peaks when gene sets are overrepresented in R3 differentially expressed genes. The heat map shows enrichment scores normalized across clusters for gene sets found enriched in R3.

FIGS. 5A-5G. Fibrotic associated macrophages include distinct subsets F1 (MHCII^{hi+}) and F2 (CD9^{hi+}IL-36γ⁺). (5A) Lineage schematic of RACs from Slingshot pseudotime analysis and trajectory including descriptive marker combination. Pseudotime trajectory is shown in a principal component plot (PC1 vs PC2). (5B) Fibrotic subsets are distinguished by specific marker profile in silico. (5C) Heat map of gene set enrichment scores normalized across clusters for gene sets found upregulated in F1 and running gene set enrichment plots for the IFNγ and IFNα responses. (5D) Gene network representation for relationships of differentially expressed genes in F1 (top) and F2 (bottom) by STRING metadata scores. (5E) Flow cytometry gating strategy specific to F1 and (F2+FP1) from F4/80+ macrophages using CD9, MHCII, CD11c. (5F) Time course of F1 and (F2+FP1) subsets in UBM, PCL and saline microenvironments (n=4 biologically independent). Two-way analysis of variance p values are presented (*p<0.05, ****p<0.0001). (5G) Immunofluorescence histology for CD9 (red) and F4/80 (green) at 1 week VML with synthetic material (scale bars=100 μm and 25 μm, respectively).

FIGS. 6A-6E. Profibrotic CD9^{hi+}IL-36γ⁺ macrophages are dependent on IL-17 signaling and terminal clusters are relevant in various pathologies. (6A) Immunofluorescent staining for mouse macrophage marker F4/80 and CD9 in wild type, Il17ra^{-/-}, and Il17a^{-/-} mice 12 weeks after implantation with PCL (scale bars=50 μm). (6B) Il36γ gene expression in wild type, Il17ra^{-/-}, and IL17a^{-/-} mice with PCL normalized to saline controls (n=4, biologically independent, ANOVA with multiple comparison, ***p<0.001). (6C) Immunofluorescent staining for CD64, CD9, IL-36γ positive macrophages in human breast implant tissue capsules (scale bars=50 μm), juvenile xanthogranuloma, and Langerhans cell histiocytosis (scale bars=200 μm). (6D) Gene expression correlations of human IL17RA with IL36γ, IL17RA and CD9 with MSR1 in human breast implant fibrotic capsules. (6E) Network diagrams and similarity heat maps for terminal fibrotic and regenerative macrophage clusters to clusters from repository single cell RNA data sets for murine models of cancer (sarcoma+/− immunotherapies aCTLA-4, aPD-1), lung fibrosis (+/− bleomycin induction) and human liver. Circles represent percent compositions of clusters by condition.

### DETAILED DESCRIPTION OF THE INVENTION

The heterogeneity of macrophages in different tissues and pathological conditions has made their phenotyping challenging, particularly in vivo. Furthermore, in vitro macrophage characterization does not adequately capture macrophage phenotypic potential due to the lack of tissue environmental signals and influence of other immune cells. By profiling single cells isolated from physiologically-relevant environments, the present inventors identified and characterized macrophages associated with diverse immune and tissue environments modelled using biomaterials. The use of scRNAseq allows for the identification of novel clusters by unsupervised clustering algorithms that is not biased by previous knowledge of the cells, providing an advantage for identifying previously unknown cell populations.

Methods of Identification of Regenerative and Fibrotic Macrophage Subtypes

The desire for more accurate macrophage phenotypic and functional characterization crosses many fields. To that end, the inventors sorted the subpopulation of macrophages (F480^{hi}/CD64^{hi}) that represent distinct polarization states as defined by canonical M1-M2 surface markers. While a continuum of macrophage phenotype has been proposed in the past, the novel unbiased classification and characterization with the single cell data of the present invention provides new phenotypic profiles that can also be identified using new surface markers and standard experimental flow cytometry and immunostaining techniques. The terminal macrophage subsets found using the methods of the present invention all expressed genes associated with M1 and M2 polarization. However, the phenotypic characterization of these subsets, using differential expression analysis, gene set enrichment, and network analysis, were consistent with known markers, showing that the new classifications of the present invention can be used to more accurately reflect macrophage behavior.

The present inventors found that macrophages associated with urinary bladder matrix tissue (UBM) and IL-4 in the tissue are heterogeneous and distributed in phenotypically distinct clusters. IL-4 is a cytokine recognized for promoting repair of muscle (4), liver (35), and cartilage (36), and is critical for macrophage polarization in a healing wound (37). The UBM environment induced greater macrophage heterogeneity with two primary terminal subsets with phenotypes relevant to tissue repair. Expression analysis of the R1 cluster suggests it is important for mobilizing and educating immune cells through the expression of chemokines and increased antigen presentation that is required during the early wound healing process. The R2 macrophages, with the highest level of Il4ra, expressed genes relevant to stimulation of other cell types important for Type 2 responses and regeneration including Ccl24 (Eotaxin-2), coding for a protein that attracts and activates eosinophils. This finding is supported by Chawla et al., that demonstrated the IL-4 secreting eosinophils are critical to muscle repair (38). The metabolic profiles of the R1 (glycolysis) and R2 (oxidative phosphorylation) correlate with distinct functions of antigen presentation and adaptive-related chemokine expression versus phagocytosis, that was validated experimentally in sorted R2 macrophages. Glycolysis has been associated with inflammatory macrophages (39) and oxidative phosphorylation with alternatively activated macrophages but macrophages (40). In vitro studies of conventional M2 macrophages required inhibition of both metabolic pathways to inhibit IL-4 induced STAT6 phosphorylation (41).

The inventors also found distribution of macrophages isolated from the PCL-treated wounds was less heterogeneous than the ECM-treated tissues and included the functional subsets F1 and F2. The F1 cluster expresses many genes associated with inflammation including interferon-related cytokines and activation of the innate and adaptive immune system. The R1 cluster also expressed markers of inflammation and mobilization but the magnitude of expression and types of inflammatory markers were significantly different. This difference in the F1 and R1 inflammatory profile suggests the importance of the early inflammatory response in directing the subsequent tissue repair or development of a foreign body capsule or fibrosis. The time course of flow cytometry revealed that R1 increased with ECM treatment. Since ECM treatment improves tissue repair, R1 may represent an inflammatory phenotype that can be targeted to enhance tissue development.

The inventors found that the F2 cluster associated with PCL treatment expressed genes that connected Type 17 immunity and markers of autoimmune disease. Type 17 immune responses are associated with autoimmunity in diseases such as psoriasis, irritable bowel syndrome and inflammatory arthritis (28, 42-44). The F2 macrophages express IL-36γ, a cytokine that is found clinically in the skin of psoriasis patients and in inflammatory arthritis (45). This cytokine was also recently identified as a target in tumors that, when blocked, enhances responsiveness to immunotherapy (46). It is also implicated in a positive feedback loop with IL-17 (28). In other work, we demonstrated that IL-17 is produced by innate lymphocytes, γδ and CD4⁺ T cells in response to PCL implantation in mice and in the fibrous capsule surrounding human breast implants (19). IL-17 is implicated in fibrotic disease in in the lung (30), heart (31), and liver (47) in addition to the foreign body response (5). The F2 macrophage population expressing IL-17 receptor A, links n IL-17 signaling, fibrosis and autoimmune disease.

Further, the inventors found F2 macrophages also expressed multiple forms of Trem (triggering receptor expressed on myeloid cells) and its ligands that are associated with autoimmune diseases such as inflammatory bowel disease and psoriasis (48, 49). TREM integrates and broadly modifies inflammatory signals across the innate and adaptive immune system. The presence of F2 surface markers and related cytokines in human tissues provides evidence that the tissue immune environment created by PCL and the mechanisms of response may be broadly relevant to various pathological conditions. Further supporting the broader relevance of the macrophage subsets, we found macrophages clusters similar to F2 in publicly available data sets of idiopathic lung fibrosis and sarcoma. Multiple genes in the F2 subset have functions that remain unknown. Based on the potential importance of this subset in disease pathology, further studies into these unknown genes may be warranted. While small in number, the F2 macrophage subset, is highly differentiated and may play a critical role in pathologies associated with the pro-inflammatory macrophages in tissue fibrosis.

In accordance with an embodiment of the present invention, all terminal macrophage populations can be experimentally identified with a combination of CD9, CD301, and CD74, so they can be readily tested in other experimental models and clinical conditions.

In accordance with an embodiment, the present invention provides a method for identification of regenerative associated macrophages in a heterogeneous cellular sample, the method comprising: contacting the heterogeneous cellular sample comprising regenerative associated macrophages, fibrotic associated macrophages, and other macrophages with a CD301 specific binding member; distinguishing the regenerative associated macrophages based on whether the CD301 cell surface marker specific binding member binds to a cell surface marker on macrophages of the sample.

As used herein, the term “regenerative associated macrophages” means a subpopulation of F480i macrophages that have been historically associated with the extracellular matrix (ECM) of tissues, and which induce tissue repair through increased expression of IL(Interleukin)-4. The regenerative associated are also associated with the traditionally-defined M2 macrophage, and which manifest a Th2 T cell response to ECM materials and have a higher CD206:CD86 surface protein expression. During the investigation, macrophages from the UBM tissue environment were identified as regenerative associated clusters (RACs).

As used herein, the term “fibrotic associated macrophages” means a subpopulation of F480i macrophages that have been historically associated with the pro-inflammatory, IFNγ activated macrophage (M1) induce fibrosis as a result of the foreign body response (FBR) that engages inflammatory M1 macrophages with significantly reduced CD206 expression resulting in higher CD86:CD206 expression. The associated macrophage phenotype occurs in a type 17 immune environment that includes innate lymphocytes, gamma-delta T cells and Th17 T cells. During the investigation, macrophages from the PCL tissue environment were identified as fibrotic associated clusters (FACs).

As detailed below, aspects of the compositions and methods include contacting a heterogeneous cellular sample with a cell surface marker specific binding member and then distinguishing a macrophage subset of interest, e.g., R1, R2, F1, or F2, for example, subset, based on binding or absence of binding to the cell surface markers specific binding members, i.e., based on whether the binding members binds to the cell surface markers. In certain aspects, the distinguishing is performed based on the binding of one or more specific binding members. For example, distinguishing may include isolating or depleting cells bound to one or more specific binding members to separate or enrich for the macrophage subset. Alternatively, or in addition, distinguishing may include identifying the macrophage subset based on one or more specific binding members bound to cells in the sample (e.g., as measured by one or more signals provided by the one or more binding members). In certain aspects, identifying the macrophage subset may enable enrichment, separation and/or assessment of the macrophage subset. For example, distinguishing may include identifying the macrophage subset and may further include providing an assessment based on a characteristic of the identified macrophage subset, such as number (relative or total), additional specific binding members bound to cells of the identified cardiomyocyte subset, and so forth. Steps of distinguishing are described in further detail below.

The heterogeneous cellular sample may include mammalian cells (e.g., human, non-human primate, murine, etc.). In certain aspects, the sample may include myocytes, such as muscle cells, or a precursor thereof. In addition, the sample may include blood cells, T-cells, B-cells, macrophages and other cells.

In certain embodiments, the cells in the sample may be live, such as when isolation of cells is desired. In other embodiments, cells of the sample may be dead (e.g., fixed and/or permeabilized for assessment of, for example, intracellular markers by fluorescence microscopy or flow cytometry), such as when only identification and/or characterization is desired.

In accordance with an embodiment, the present invention provides a method for identification of a subpopulation of regenerative associated macrophages known as R1 macrophages in a heterogeneous cellular sample, the method comprising: contacting a sample of the regenerative associated macrophages that were distinguished with a CD301 specific binding member binding to the macrophages above with a CD9 and MHCII specific binding member; distinguishing the R1 macrophages based on whether the CD9 and MHCII surface marker specific binding members both bind to the cell surface markers on macrophages of the sample.

As used herein, regenerative associated macrophages known as R1 are defined as being CD301b+, and CD9+MHCII+. R1 macrophages are upregulated in genes for Cd74 (coding MHCII), Ccr2, Il1b, and Gapdh. R1 macrophages are enriched in leukocyte activation gene sets, suggesting that these macrophages play a role in communication and activation of the adaptive immune system, and express gene modules associated with glycolysis (Enol, Gapdh), antigen presentation (H2 genes and Cd74), and inflammatory cytokines (Cxcl1, Ccr2, Ccl5, Tnfa, and Il1b).

In accordance with an embodiment, the present invention provides a method for identification of a subpopulation of regenerative associated macrophages known as R2 macrophages in a heterogeneous cellular sample, the method comprising: contacting a sample of the regenerative associated macrophages that were distinguished with a CD301 specific binding member binding to the macrophages above with a CD9 and MHCII specific binding member; distinguishing the R2 macrophages based on whether neither of the CD9 and MHCII surface marker specific binding members bind to the cell surface markers on macrophages of the sample.

As used herein, regenerative associated macrophages known as R2 are defined as being CD301b+, CD9-MHCII−. R2 macrophages are upregulated in genes for associated with a classical alternatively-activated or anti-inflammatory macrophage. R2 macrophages are enriched in classical anti-inflammatory genes such as Chil3, Cd163, and Mrc1 (gene encoding CD206). Gene set enrichment and gene modules from network analysis support a unique metabolic profile with expression of Cox5a, Uqcrq, Ndufa1, and Ndufc2. This profile supports R2 activation of oxidative phosphorylation compared to glycolysis in R1. R2 macrophage expression also included gene set enrichment and endocytic gene modules (Cltc, Clta, and Ap2a2) that suggest phagocytic activity in this subset.

Metabolism is recognized as a defining feature of different immune programs and as a potential target in regulating immune function. Glycolysis has long been associated with inflammatory macrophages and oxidative phosphorylation with alternatively activated macrophages but the single subsets present additional metabolic complexity. In vitro studies of M2 macrophages that would include both R1 and R2 based on traditional profiling support the presence of both oxidative-phosphorylation and glycolysis metabolic pathways in RACS. Inhibition of both metabolic pathways was required to inhibit IL-4-induced STAT6 phosphorylation (Wang et al, Cell metabolism).

Oxidative phosphorylation is an enzymatic process that occurs in both prokaryotes and eukaryotes. In eukaryotes, the process occurs as part of cellular respiration within the mitochondrion. In prokaryotes, it occurs in the cell membrane itself. This process is a more efficient method to produce ATP (in terms of net ATP yield) than fermentation. The process however involves oxidation that it produces reactive oxygen species, which contributes to the propagation of free radicals.

Oxidative phosphorylation is carried out through a series of compounds in a chain called the electron transport chain. In this chain, electron is transferred from one compound to another via redox reactions. It is coupled with the transfer of proton (H+ ion) across the membrane resulting in the creation of a proton gradient, which is essential in the synthesis of energy-storing compounds, e.g. ATP. Thus, the electron transport chain is a crucial cellular machinery for its major role in extracting energy via redox reactions in cellular respiration as well as in photosynthesis. The electron transport chain is comprised chiefly of electron donors and acceptors. The final electron acceptor is an oxygen molecule, which makes it an aerobic process.

The unique metabolic profiles of the R1 (glycolysis) and R2 (oxidative phosphorylation) correlate with distinct functions of antigen presentation and adaptive-related chemokine expression found using the methods of the present invention versus phagocytosis. The inflammatory F1 macrophages that were active in interferon responses did not have unique metabolic signatures compared to the other subsets and were distinct from R1.

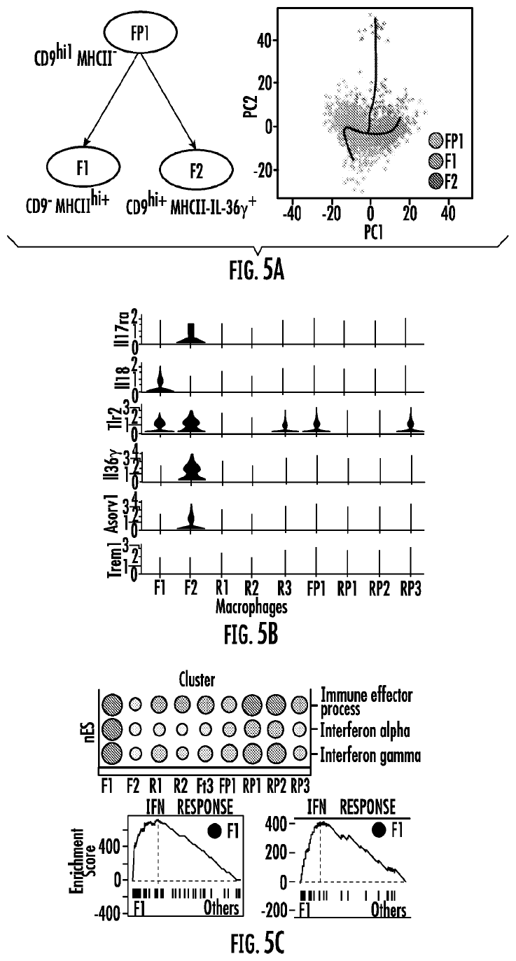

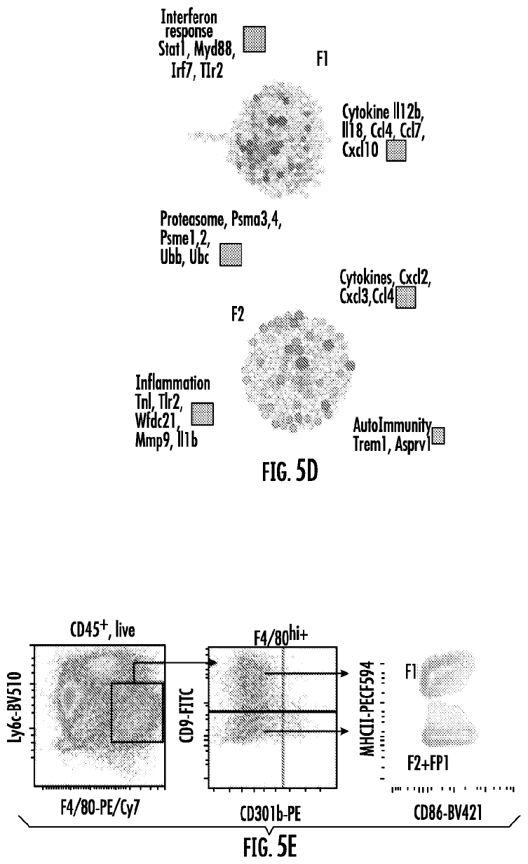

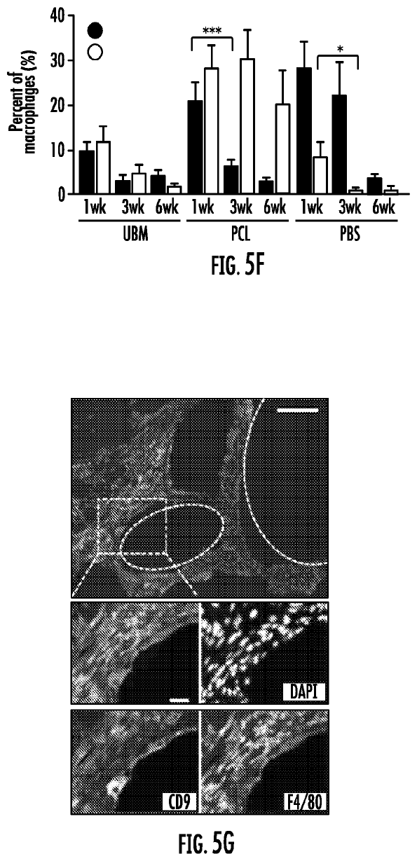

Fibrotic-associated macrophages were first defined as CD301b-macrophages (the marker for RACs). Then F1 macrophages were isolated and identified as CD9-MHCII+. The F1 cluster of macrophages express traditional markers of inflammation and genes associated with the interferon response including Irf7, Irf8, and Tlr2 (FIG. 5B). Gene set enrichment of F1 was upregulated for both IFNα and IFNγ response (FIG. 5C). Network analysis showed modules associated with interferon response (Stat1, Myd88, Irf7, and Tlr2) and cytokines associated with inflammatory function (Il18, Ccl4, Ccl7, and Cxcl10).

In accordance with an embodiment, the present invention provides a method for identification of fibrotic associated macrophages in a heterogeneous cellular sample, the method comprising: contacting the heterogeneous cellular sample comprising regenerative associated macrophages, fibrotic associated macrophages, and other macrophages with a CD301 specific binding member; distinguishing the fibrotic associated macrophages based on whether the CD301 cell surface marker specific binding member does not bind to a cell surface marker on macrophages of the sample.

In accordance with an embodiment, the present invention provides a method for identification of a subpopulation of fibrotic associated macrophages known as F1 macrophages in a heterogeneous cellular sample, the method comprising: contacting a sample of the fibrotic associated macrophages that were distinguished with a CD301 specific binding member which did not bind to the macrophages in the previous paragraph with a CD9 and MHCII specific binding member; distinguishing the F1 macrophages based on whether the CD9 surface marker specific binding member does not bind to the cell surface markers and the MHCII specific binding member does bind to the cell surface markers on macrophages of the sample.

In accordance with an embodiment, the present invention provides a method for identification of a subpopulation of fibrotic associated macrophages known as F2 macrophages in a heterogeneous cellular sample, the method comprising: contacting a sample of the fibrotic associated macrophages that were distinguished with a CD301 specific binding member which did not bind to the macrophages in the previous paragraph with a CD9 and MHCII specific binding member; distinguishing the F2 macrophages based on whether the CD9 surface marker specific binding member binds to the cell surface markers and the MHCII specific binding member does not bind to the cell surface markers on macrophages of the sample.

In accordance with an embodiment, F2 macrophages are defined as fibrotic-associated macrophages which are CD301b-macrophages, which are also CD9+MHCII−. F2 macrophages express standard inflammatory markers Slpi, Hdc, Tlr2, and Il1b they also expressed genes associated with autoimmunity S100a8, S100a9 (Calprotectin), Il36γ, Trem1, and Asprv1.

The macrophages associated with a fibrotic environment include F1 that expressed a typical inflammatory profile centered on interferon and associated response elements. F2 however is a unique fibrotic macrophage subset that most directly demonstrates the advantages of performing single cell analysis on the sorted macrophage population. The F2 population is small and thus may not have been identified in a broader immune population analysis. Even after sorting the CD64⁺F4/80⁺ macrophage subpopulation, F2 consisted of 1% of the total isolated cells. As the sorted macrophages consist of ˜20% of all CD45⁺ cells, even sorting on CD45⁺ would make F2˜0.2% of the total population. This macrophage population presents multiple new genes and targets for consideration in tissue fibrosis that also connect Type 17 and autoimmunity. Type 17 immune responses are connected to autoimmunity in diseases such as psoriasis and inflammatory arthritis. Il36γ is found in the skin of psoriasis patients and in inflammatory arthritis (Carrier, Ma et al. 2011, Zambrano-Zaragoza, Romo-Martinez et al. 2014). IL-17 has been implicated in multiple fibrotic diseases including fibrosis in the lung (Wilson, Madala et al. 2010), heart (Wu, Ong et al. 2014) and liver (Seo, Eun et al. 2016) in addition to the foreign body response (Wynn and Vannella 2016) but there was no association with autoimmunity. The F2 macrophage cluster links together a Type 17 response and autoimmunity in fibrosis more broadly. Furthermore, multiple genes in the F2 subset have functions that remain unknown. While small in number, the F2 macrophage subset, is highly differentiated from the interferon-focused F1 and may play a critical role in pathologies associated with the pro-inflammatory macrophages in tissue fibrosis

In certain embodiments, the method may include steps of obtaining and/or culturing the cell sample prior to the step of contacting. For example, the method may include obtaining cells from a subject's blood, bone marrow, skin, heart, liver, stomach, and so forth. Cells obtained from any of the above sources may be further purified (e.g., enriched) based on morphology, surface marker expression and/or by continued passage.

Contacting the Heterogeneous Cellular Sample with A Cell Surface Specific Binding Member(S).

Aspects of the invention include samples of the above embodiments contacted with one or more specific binding members (e.g., as described below). As summarized above, the method includes contacting a heterogeneous cellular sample with a cell surface marker specific binding member. The cell surface marker specific binding member may be a lymphocyte cell surface specific binding member. In certain aspects, the lymphocyte cell surface specific binding member may be a CD301 specific binding member. CD301, also known as CLEC10A, macrophage galactose/N-acetylgalactosamine (GalNAc) specific lectin (MGL), DCASGPR, and HML, is a 40 kD type II transmembrane glycoprotein, which belongs to the C-type lectin family. Human CD301 consists of a 39 amino acid (aa) cytoplasmic region, a 21 aa transmembrane segment, and a 256 aa extracellular domain (ECD) with one carbohydrate recognition domain (CRD) and a neck region. CD301 is expressed on immature myeloid dendritic cells and alternatively activated (tolerogenic) macrophages. The expression level is upregulated by immunosuppressant dexamethasone. Human CD301 has an exclusive specificity for rare terminal GalNAc structures, which are revealed on the tumor-associated mucin MUC1 and CD45 (RA, RB, and RC but not RO isoforms). This interaction inhibits effector T cell activation and induces their apoptosis. CD301 also binds the GP envelope glycoprotein on Marburg and Ebola viruses and enhances viral entry and infectivity.

CD9 is a 24 kD type III transmembrane protein also known as tetraspanin, MRP-1 and DRAP-24. It is a member of the tetraspan family (spanning the membrane four times) found on platelets, B cell progenitors, activated lymphocytes, granulocytes, endothelial cells and epithelial cells. CD9 induces adhesion, platelet aggregation, and B cell development. CD9 has been shown to associate with CD63, CD81, CD82, and CD36 and to bind to 31 integrins.

CD74 is a type II transmembrane glycoprotein also known as MHC class II associated invariant chain (MHCII), invariant chain, Ii, MHC class II chaperone, and MIF receptor. CD74 exists in four isoforms with molecular masses of 33, 35, 41, and 43 kD, depending on genetic splicing. CD74 is primarily expressed on antigen presenting cells, including B cells, monocytes/macrophages, dendritic cells, and Langerhans cells. It is also expressed by activated T cells and activated endothelial and epithelial cells as well as carcinomas of lung, renal, gastric and thymic origin. The primary function of CD74 is intracellular sorting of MHC class II molecules and regulation of exogenous peptide loading onto MHC class II. It is also involved in the modulation of B cell differentiation and positive selection of CD4+ T cells. It has been reported that CD74 binds MIF (macrophage migration inhibitory factor) and signals through CD44 to regulate innate and adaptive immunity. It is also reported that H. pylori infection occurs through urease B binding of CD74 on gastric epithelial cells, inducing gastric epithelial cell apoptosis, NF-κB activation, and IL-8 production.

A variety of specific binding members are suitable for embodiments of the subject invention. In any of the above embodiments, one or more of the specific binding members may include a specific binding domain. In certain aspects, the specific binding domain may be an antibody or a fragment thereof. The specific binding member may also include a processing domain, such as a solid support or a detectable label, e.g., as described in greater detail below. In some instances, the specific binding member is a non-naturally occurring specific binding member. For example, the specific binding member may include a processing domain, such as described below, that is not naturally present in a specific binding member, such as a naturally occurring antibody. In some instances, the specific binding member may be part of a specific binding member composition that is not naturally occurring, e.g., a composition in which there is only a single type of the specific binding member in multiple copies (e.g., monoclonal antibody composition), a composition in which the specific binding member is present in a non-naturally occurring medium, such as a buffered medium that lacks one or more components found in the naturally occurring medium (e.g., blood) of the specific binding member (for example, the specific binding member may be present in a composition that lacks cellular components or blood proteins), etc.

In certain embodiments, a specific binding member may include a specific binding domain. The terms “specific binding”, “specific for”, “specifically binds” and the like, refer to the preferential binding of the binding member to a particular target (e.g., to a cell type, to a specific extracellular or intracellular marker, etc.). The specific binding domain may bind (e.g., covalently or non-covalently) to a specific epitope on or within the cell. In certain aspects, a specific binding domain non-covalently binds to a target. In such instances, the specific binding domain association with the binding target (e.g., CD301, CD9, MHCII) may be characterized by a KD (dissociation constant) of 10⁻⁵ M or less, 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less. A variety of different types of specific binding domains may be employed. Specific binding domains of interest include, but are not limited to, antibodies, proteins, peptides, haptens, nucleic acids, etc. The term “antibody” as used herein includes polyclonal or monoclonal antibodies or fragments that are sufficient to bind to a target of interest. The term “antibody” also includes antibody fragments, such as, monomeric Fab fragments, monomeric Fab′ fragments, or dimeric F(ab)′2 fragments. Also within the scope of the term “antibody” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies.

As used herein, “antibody” includes reference to an immunoglobulin molecule immunologically reactive with one or more particular antigens on TGF-β, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)2, Fab, Fv and rIgG. See also, Pierce Catalog and Handbook, 1994-1995 (Pierce Chemical Co., Rockford, Ill.). See also, e.g., Kuby, J., Immunology, 3.sup.rd Ed., W.H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol:5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form. Also, the antibody can have any level of affinity or avidity for the functional portion of IL-17 or IL-36γ, or any portion or fragment which inhibits IL-17 or IL-36γ binding the appropriate receptor. Desirably, the antibody is specific for the functional portion of IL-17 or IL-36γ, or any portion or fragment which inhibits IL-17 or IL-36γ binding the appropriate receptor, such that there is minimal cross-reaction with other peptides or proteins.

Methods of testing antibodies for the ability to bind to any for the functional portion of IL-17 or IL-36γ, or any portion or fragment which inhibits IL-17 or IL-36γ binding the appropriate receptor are known in the art and include any antibody-antigen binding assay, such as, for example, radioimmunoassay (RIA), ELISA, Western blot, immunoprecipitation, and competitive inhibition assays (see, e.g., Janeway et al., infra, and U.S. Patent Application Publication No. 2002/0197266 A1).

As mentioned above, one or more specific binding members of the subject embodiments may include (or be conjugated to) a processing domain. By processing domain is meant any suitable domain (e.g., molecule, structure, etc.) for identifying the presence of the specific binding member (such as a label domain), separating cells bound by the specific binding member (such as a solid support), or both.

In certain aspects, separation or enrichment, e.g., as described below, may be performed using specific binding members that are conjugated to a solid support. The solid support may be any suitable solid support, such as the interior surface of a container (e.g., flask, tube, well, etc.) or a microparticle. For example, the solid support may be a magnetic microparticle, and separation may include magnetically separating or removing cells bound to the microparticle from unbound cells.

In certain embodiments, the processing domain may be a label domain. For example, the specific binding member may be detectably labeled with a fluorophore. The label domain may be a colored dye, a phosphorescent label, a fluorescent label, a mass tag, a radioactive label, or any other suitable label. For example, the label domain may be a fluorescent label detectible based on, for example, fluorescence emission maxima, fluorescence polarization, fluorescence lifetime, light scatter, or a combination thereof. In certain aspects, the label domain may be a fluorophore (i.e., a fluorescent label, fluorescent dye, etc.). Fluorophores can be selected from any of the many dyes suitable for use in analytical applications (e.g., flow cytometry, imaging, etc.). A large number of dyes are commercially available from a variety of sources, such as, for example, Molecular Probes (Eugene, Oreg.) and Exciton (Dayton, Ohio). Examples of fluorophores that may be incorporated into the microparticles include, but are not limited to, 4-acetamido-4′-isothiocyanatostilbene-2,2′ disulfonic acid; acridine and derivatives such as acridine, acridine orange, acridine yellow, acridine red, and acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy5, Cy5.5, and Cy7; 4′,6-diaminidino-2-phenylindole (DAPI); 5′, 5″-dibromopyrogallol-sulfonphthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Green Fluorescent Protein (GFP); Reef Coral Fluorescent Protein (RCFP); Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; or combinations thereof. Other fluorophores or combinations thereof known to those skilled in the art may also be used, for example those available from Molecular Probes (Eugene, Oreg.) and Exciton (Dayton, Ohio). The fluorescent label may be distinguishable based on fluorescence emission maxima, and optionally further based on light scatter or extinction.

In other aspects, the label domain may be a metal isotope detectible by mass spectroscopy, such as by the time of flight mass spectrometer used in mass cytometry, e.g., as described in international patent application serial no. PCT/US2012/020950 published as WO/2010/097070, the disclosure of which is herein incorporated by reference.

As discussed above, aspects of the methods include distinguishing a macrophage subset in a heterogeneous cellular sample. Distinguishing may include separating, enriching, identifying, providing an assessment, among other protocols in which the target macrophage subset of interest is treated in a way that is different or distinct from the treatment of other cellular constituents of the heterogeneous cellular sample. In certain aspects, distinguishing may include separating cells based on binding to the surface marker specific binding member. For example, the method may include separating (e.g., isolating) cells bound to a macrophage specific binding member (such as a CD301 specific binding member).

Distinguishing may include identifying cells (e.g., based upon a cell surface signature). In certain embodiments, distinguishing may include identifying cells based upon the presence of a signal (e.g., a fluorescence signal as described above) provided by the cell surface specific binding member (e.g., a macrophage specific binding member such as a CD301 specific binding member) and optionally one or more signals provided by one or more additional binding members (e.g., CD9, and/or MHCII specific binding members, etc.).

In any of the above embodiments, the threshold value may be determined based upon input from a user or may be based upon a standardized control. In one example, the standardized control may be control particles, such as fluorescent control beads or control cells. The control particles may serve as a positive or negative control. Alternatively, or in addition, the threshold value may be determined by an algorithm configured to cluster and/or otherwise separate cell populations based on a signal, such as a signal provided by the macrophage specific binding member(s).

In addition, the distinguishing may include providing an assessment of one or more characteristics of cells identified by any of the above embodiments. For example, the assessment may include a number of the identified cells, or a relative number of the identified cells (e.g., a ratio, percentage, etc., of the identified cells to the number of cells in the sample or number of a subset of cells in the sample). Alternatively, or in addition, the assessment may include the amount of (e.g., signal obtained from, average signal obtained from, etc.) the macrophage specific binding member bound to the identified cells.

Distinguishing may include providing an assessment (e.g., to a user or operator of the method) based upon any of the above-mentioned characteristics of the identified cells. For example, the sample may be an aliquot from a cell culture, and distinguishing may include providing an assessment of whether the cell culture is suitable for use in a drug screen (e.g., such as when the number or relative number of the identified cells is above a predetermined threshold). In another example, the sample may be an aliquot from a subject, and distinguishing may include providing an assessment of whether the sample shows a regenerative or fibrotic population of macrophages. In an example, the assessment may be a recommendation of whether an implant or drug administered to cells of the sample is suitable for use in the treatment of a tissue related disease or transplant.

In some embodiments, the assessment may be provided by providing, i.e. generating, a written report that includes the artisan's assessment. Thus, a subject method may further include a step of generating or outputting a report providing the results of an assessment, which report can be provided in the form of an electronic medium (e.g., an electronic display on a computer monitor), or in the form of a tangible medium (e.g., a report printed on paper or other tangible medium).

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a patient data section, including patient medical history (which can include, e.g., age, race, serotype, etc.), as well as administrative patient data such as information to identify the patient (e.g., name, patient date of birth (DOB), gender, mailing and/or residence address, medical record number (MRN), room and/or bed number in a healthcare facility), insurance information, and the like), the name of the patient's physician or other health professional who ordered the monitoring assessment and, if different from the ordering physician, the name of a staff physician who is responsible for the patient's care (e.g., primary care physician).

The report may include a sample data section, which may provide information about the biological sample analyzed in the assessment, such as the source of biological sample obtained from the patient (e.g. blood, saliva, or type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu). The report may include an assessment report section, which may include information generated after processing of the data as described herein. The assessment portion of the report can optionally also include a recommendation(s).

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

In certain aspects, one or more of the above steps (e.g., identifying, separating, assessing, etc.) may be performed by flow cytometry. Flow cytometry is a methodology using multi-parameter data for identifying and distinguishing between different particles, such as cells or beads, that vary from one another (e.g., in terms of label, size, granularity, etc.) in a fluid medium. In flow cytometrically analyzing the particles (e.g., the cells prepared as described above), a liquid medium including the particles is first introduced into the flow path of the flow cytometer. When in the flow path, the particles are passed substantially one at a time through one or more sensing regions, where each of the particles is exposed individually to a source of monochromatic light and measurements of light scatter parameters and/or fluorescent emissions as desired (e.g., two or more light scatter parameters and measurements of one or more fluorescent emissions) are separately recorded for each particle. The data recorded for each particle is analyzed in real time or stored in a data storage and analysis means, such as a computer, as desired.

More specifically, in a flow cytometer, the particles are passed, in suspension, substantially one at a time in a flow path through one or more sensing regions where in each region each particle is illuminated by an energy source. The energy source may include an illuminator that emits light of a single wavelength, such as that provided by a laser (e.g., He/Ne or argon) or a mercury arc lamp with appropriate filters. For example, light at 488 nm may be used as a wavelength of emission in a flow cytometer having a single sensing region. For flow cytometers that emit light at two distinct wavelengths, additional wavelengths of emission light may be employed.

In series with a sensing region, a detector module that includes one or more detectors, e.g., light sensors, such as photomultiplier tubes (or “PMT”), is used to record light that passes through each particle (generally referred to as forward light scatter), light that is reflected orthogonal to the direction of the flow of the particles through the sensing region (generally referred to as orthogonal or side light scatter) and fluorescent light emitted from the particles, if it is labeled with fluorescent marker(s), as the particle passes through the sensing region and is illuminated by the energy source. The forward light scatter (or FSC), orthogonal light scatter (SSC), and each fluorescence emissions include a separate parameter for each particle (i.e. each “event”). Thus, for example, two, three four or more parameters can be collected (and recorded) from a particle labeled with two different fluorescence markers.

Flow cytometers further include data acquisition, analysis and recording means, such as a computer, wherein multiple data channels record data from each detector for the light scatter and fluorescence emitted by each particle as it passes through the sensing region. The purpose of the analysis system is to classify and count particles wherein each particle presents itself as a set of digitized parameter values. In the flow cytometry assay methods of the invention, the flow cytometer may be set to trigger on a selected parameter in order to distinguish the particles of interest from background and noise. “Trigger” refers to a preset threshold for detection of a parameter. It is typically used as a means for detecting passage of particle through the laser beam. Detection of an event that exceeds the preset threshold for the selected parameter triggers acquisition of light scatter and fluorescence data for the particle. Data is not acquired for particles or other components in the medium being assayed which cause a response below the threshold. The trigger parameter may be the detection of forward scattered light caused by passage of a particle through the light beam. The flow cytometer then detects and collects the light scatter and fluorescence data for particle.

A particular subpopulation of interest may be further analyzed by “gating” (i.e. a type of threshold) based on the data collected for the entire sample. To select an appropriate gate, the data may be plotted (e.g., on a linear or logarithmic scale) so as to obtain the best separation of subpopulations possible. This procedure is typically done by plotting forward light scatter (FSC) vs. side (i.e., orthogonal) light scatter (SSC) on a two-dimensional dot plot. The flow cytometer operator then selects the desired subpopulation of particles (i.e., those cells within the gate) and excludes particles which are not within the gate. Where desired, the operator may select the gate by drawing a line around the desired subpopulation using a cursor on a computer screen. Only those particles within the gate are then further analyzed by plotting the other parameters for these particles, such as fluorescence. Gating based on fluorescence may then be used to further separate subpopulations of cells,

Aspects of the invention further include systems for use in practicing embodiments of the subject methods. Systems of the invention may include a flow cytometer configured to assay particles (e.g., beads, cells, etc.) by measuring signals such as FSC, SSC, fluorescence emission maxima, light scatter, mass, molecular mass, etc. Aspects of the system include a flow channel, a detector module configured to obtain a signal from a detectably labeled macrophage specific binding member when present in an assay region of the flow channel, and a signal processing module configured to identify a macrophage subset based on a signal produced by the detectably labeled macrophage specific binding member. In certain aspects, the macrophage specific binding member may be a CD301, CD9, or MHCII specific binding member. The flow channel may include a heterogeneous cellular sample that includes the macrophage specific binding member.

The signal processing module may be configured to perform any of the method steps of distinguishing (e.g., identifying, sorting, providing an assessment, etc.) such as described above. In certain embodiments, the signal processing module may be configured to identify a cell as belonging to a macrophage subset of interest (such as R2 macrophages) when the intensity of the signal obtained from the cell is above a predetermined threshold. Alternatively, or in addition, the signal processing module may be configured to identify a cell as belonging to the macrophage subset (such as R1 macrophages) when the intensity of the signal obtained from the cell is below a predetermined threshold. The signal processing module may be configured to separate the identified macrophage subset from other cells in the sample. Alternatively, or in addition, the signal processing module may be configured to provide an assessment of the sample, e.g., based on the relative number of cells in the identified macrophage subset, the strength of the signal obtained from the identified macrophage subset, or a combination thereof. In certain aspects, the assessment may include a determination of whether a cell culture is suitable for use in a drug screen.

The system may be a flow cytometric system. Flow cytometers of interest include, but are not limited to, devices such as those described in U.S. Pat. Nos. 4,704,891; 4,727,029; 4,745,285; 4,867,908; 5,342,790; 5,620,842; 5,627,037; 5,701,012; 5,895,922; and 6,287,791; the disclosures of each of which are herein incorporated by reference. The system may be configured to separate particles (e.g., cells or beads) into separate containers (e.g., one or more tubes, waste, etc.) based on a one or more light scatter and signals obtained from the particle.

The system can in certain embodiments include a computer that includes: a central processing unit; a main non-volatile storage drive, which can include one or more hard drives, for storing software and data, where the storage drive is controlled by disk controller; a system memory, e.g., high speed random-access memory (RAM), for storing system control programs, data, and application programs, including programs and data loaded from non-volatile storage drive; system memory can also include read-only memory (ROM); a user interface, including one or more input or output devices, such as a mouse, a keypad, and a display; an optional network interface card for connecting to any wired or wireless communication network, e.g., a printer; and an internal bus for interconnecting the aforementioned elements of the system.

Operation of computer is controlled primarily by operating system, which is executed by central processing unit. The operating system can be stored in a system memory. In some embodiments, the operating system may include a file system. In addition to an operating system, one possible implementation of the system memory includes a variety of programming files and data files for implementing the method described below. In certain cases, the programming can contain a program, where the program can be composed of various modules, and a user interface module that permits a user at user interface to manually select or change the inputs to or the parameters used by programming. The data files can include various inputs for the programming.

The memory of a computer system can be any device that can store information for retrieval by a processor, and can include magnetic or optical devices, or solid state memory devices (such as volatile or non-volatile RAM). A memory or memory unit can have more than one physical memory device of the same or different types (for example, a memory can have multiple memory devices such as multiple drives, cards, or multiple solid state memory devices or some combination of the same). With respect to computer readable media, “permanent memory” refers to memory that is permanent. Permanent memory is not erased by termination of the electrical supply to a computer or processor. Computer hard-drive ROM (i.e., ROM not used as virtual memory), CD-ROM, flash drives, and solid state drives are all examples of permanent memory. Random Access Memory (RAM) is an example of non-permanent (i.e., volatile) memory. A file in permanent memory can be editable and re-writable.

In certain embodiments, instructions in accordance with any of the methods described herein can be coded onto a computer-readable medium in the form of “programming”, where the term “computer readable medium” as used herein refers to any storage or transmission medium that participates in providing instructions and/or data to a computer for execution and/or processing. Examples of storage media include a hard disk, optical disk, magneto-optical disk, CD-ROM, CD-R, non-volatile memory card, ROM, DVD-ROM, flash drive, solid state disk, and network attached storage (NAS), whether or not such devices are internal or external to the computer. A file containing information can be “stored” on computer readable medium, where “storing” means recording information such that it is accessible and retrievable at a later date by a computer.

In certain embodiments, the method may include steps of obtaining and/or culturing the cell sample prior to the step of contacting. For example, the method may include obtaining cells from a subject's blood, bone marrow, skin, heart, liver, stomach, and so forth. Cells obtained from any of the above sources may be further purified (e.g., enriched) based on morphology, surface marker expression and/or by continued passage.

For isolation of cells from tissue, an appropriate solution may be used for dispersion or suspension. The solution may be a balanced salt solution, e.g., normal saline, PBS, Hank's balanced salt solution, etc., conveniently supplemented with fetal calf serum, human platelet lysate or other factors, in conjunction with an acceptable buffer at low concentration, such as from 5-25 mM. Convenient buffers include HEPES, phosphate buffers, lactate buffers, etc. The separated cells may be collected in any appropriate medium that maintains the viability of the cells. Various media/buffers are commercially available and may be used according to the nature of the cells, including dMEM, HBSS, dPBS, RPMI, Iscove's medium, etc., frequently supplemented with fetal calf serum or human platelet lysate.

Kits

In yet another aspect, the present invention provides kits for practicing the subject methods, e.g., as described herein. The kit may include a macrophage specific binding member having a macrophage specific binding domain coupled to a first processing domain. Alternatively, or in addition, the kit may include a macrophage specific binding member and/or an intracellular marker specific binding member. The processing domains each may include a solid support (such as a microparticle, magnetic microparticle, etc.) or a label domain as described in the subject methods.

In certain aspects, the kit may contain any suitable reagents for maintaining pluripotency and/or cell viability, such as fetal calf serum, human platelet lysate, and so forth.

The kit may further include reagents for performing a flow cytometric assay. Examples of said reagents include buffers for at least one of reconstitution and dilution of the first and second detectible molecules, buffers for contacting a cell sample with one or both of the first and second detectible molecules, wash buffers, control cells, control beads, fluorescent beads for flow cytometer calibration and combinations thereof. In certain aspects, the kit may include one or more standardized controls. The standardized controls may be control particles such as control beads or control cells.

The specific binding members and/or reagents described above may be provided in liquid or dry (e.g., lyophilized) form. Any of the above components (detectible labels and/or reagents) may be present in separate containers (e.g., separate tubes, bottles, or wells in a multi-well strip or plate). In addition, one or more components may be combined into a single container, e.g., a glass or plastic vial, tube or bottle.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., a hard disk, optical disk, magneto-optical disk, CD-ROM, CD-R, non-volatile memory card, ROM, DVD-ROM, flash drive, solid state disk, etc., on which the information has been recorded. Yet another means that may be present is a website address which may be used via the internet to access the information at a removed site. Any convenient means may be present in the kits.

Methods of Treatment and Prevention of Fibrosis and Diseases Associated with Fibrosis

In accordance with an embodiment, the present invention provides a method for reducing or treating the progression of a fibrosis associated disease or condition in a subject in need thereof, comprising reducing or inhibiting a cell or population of cells expressing the F1 and/or F2 macrophage subtype in the subject.

As used herein, the terms “fibrosis associated disease or condition” encompass a wide spectrum of clinical entities including systemic fibrotic diseases such as systemic sclerosis (SSc), sclerodermatous graft vs. host disease, and nephrogenic systemic fibrosis, as well as numerous organ-specific disorders including radiation-induced fibrosis and cardiac, pulmonary, liver, and kidney fibrosis. Although their causative mechanisms are quite diverse and in several instances have remained elusive, these diseases share the common feature of an uncontrolled and progressive accumulation of fibrotic tissue in affected organs causing their dysfunction and ultimate failure. Some specific examples of fibrosis associated disease include, but are not limited to, multifocal fibrosclerosis (IgG₄-associated fibrosis), Nephrogenic systemic fibrosis, Hypertension-associated cardiac fibrosis, Post-myocardial infarction, Chagas disease-induced myocardial fibrosis, Diabetic and hypertensive nephropathy, Urinary tract obstruction-induced kidney fibrosis, Inflammatory/autoimmune-induced kidney fibrosis, Aristolochic acid nephropathy, Polycystic kidney disease, Idiopathic pulmonary fibrosis, Silica-induced pneumoconiosis (silicosis), Asbestos-induced pulmonary fibrosis (asbestosis), Chemotherapeutic agent-induced pulmonary fibrosis, Alcoholic and nonalcoholic liver fibrosis, Hepatitis C-induced liver fibrosis, Primary biliary cirrhosis, Parasite-induced liver fibrosis (schistosomiasis), Radiation-induced fibrosis (various organs), Bladder fibrosis, Intestinal fibrosis, Peritoneal sclerosis, Diffuse fasciitis, Localized scleroderma, keloids, Dupuytren's disease, Peyronie's disease, Myelofibrosis, and Oral submucous fibrosis.

As used herein, a “wound” refers to a physical disruption of the continuity or integrity of a tissue structure. “Wound healing” refers to the process of restoring the integrity of the tissue. The skilled artisan will understand that this may refer to a partial or a full restoration of tissue integrity. Accordingly, treatment of a wound refers to the promotion, improvement, progression, or acceleration of one or more stages or processes associated with the wound healing process. The wound may be acute or chronic. Chronic wounds may simply be described as wounds that fail to heal on a normal timeframe, which may vary depending on the nature of the wound and the specific tissue affected. The wound may also be any internal wound, e.g. where the external structural integrity of the skin is maintained, but the integrity of an underlying tissue/structure is disrupted.

In some embodiments, the fibrosis associated disease is the foreign body reaction or response. The foreign body reaction comprises the actions of macrophages and foreign body giant cells, and is the end-stage response of the inflammatory and wound healing responses following implantation of a medical device, prosthesis, or biomaterial. Typical events in the foreign body reaction include protein adsorption, monocyte/macrophage adhesion, macrophage fusion to form foreign body giant cells, consequences of the foreign body response on biomaterials, and cross-talk between macrophages/foreign body giant cells and inflammatory/wound healing cells.

In some aspects, the present inventive methods are directed to inhibition or prevention of the foreign body reaction to a surgical implantation of a medical device, prosthesis, or biomaterial. The inhibition or prevention can comprise administration of small molecules or biologically active agents which reduce or inhibit fibrosis associated macrophage subtypes, including, for example, F1 and F2 subtypes as described herein.

As used herein, the terms “reducing or inhibiting cells expressing the F1 and/or F2 macrophage subtype” means any method or process which either inhibits generation of macrophages having the F1 and/or F2 macrophage subtype as described herein, or otherwise suppresses further activation of macrophages having the F1 and/or F2 macrophage subtype either locally at the site of tissue injury or wound, or systemically.

As used herein, the terms “tissue injury or wound” means any trauma, disease, chemical or other environmental exposures which damage tissue. Tissue injury may result from many different causes. For example, tissue injury may occur following ischemia, hemorrhage, trauma, surgery, transplantation, inflammation, infection, burns, disease progression, aging, surgical implantation of a medical device, prosthesis, or biomaterial, and many other causes.

In accordance with another embodiment, the present invention provides a method for reducing or inhibiting a cell or population of cells expressing the F1 and/or F2 macrophage subtype in a subject in need thereof, comprising administering to the subject an effective amount of an IL-17 and/or IL36γ inhibitor.

IL-17A is a pro-inflammatory cytokine. It belongs to the IL-17 family, which consists of IL-17A-F. IL-17A plays a role in neutrophil recruitment, host defense and immuno-inflammatory pathology. It is secreted mainly by Th17, but also by Treg cells, NK cells, mast cells and neutrophils. IL-17A and IL-17F bind to the same receptor, however the influence of IL-17A on gene regulation is 10-30 times stronger. The function of IL-17B, IL-17C and IL-17D is poorly defined. IL-17E limits Th17 development and promotes Th2 cytokines

As used herein, the term “IL-17” inhibitor means a small molecule, antibody or functional portion or fragment thereof, proteins, peptides, siRNAs, antagonists, agonists, compounds, or nucleotide constructs which either reversibly or irreversibly bind IL-17A-F and prevent its binding to a IL-17 receptor on a cell or tissue in a subject. The term can also mean a small molecule, antibody or functional portion or fragment thereof, proteins, peptides, siRNAs, antagonists, agonists, compounds, or nucleotide constructs which either reversibly or irreversibly bind IL-17 receptors in an antagonistic manner such that IL-17A-F and its analogs or derivatives cannot stimulate the IL-17 receptors in cells and tissues in a subject. Examples of IL-17 inhibitors include, but are not limited to, secukinumab, a fully human anti-IL-17A monoclonal antibody, brodalumab is a human, anti-IL17RA monoclonal antibody. It blocks the activity of IL17RA, 17A/F and 17E. Ixekizumab is a humanized IgG₄ monoclonal antibody that neutralizes IL-17. Other antibodies to IL-17 include bimekizumab, ALX-0761, CJM112, CNTO 6785, LY3074828, and SCH-900117.

Interleukin-36γ is a cytokine previously known as interleukin-1 family member 9 (IL1F9). IL-36γ is a protein that in humans is encoded by the IL36G gene. The protein encoded by this gene is a member of the interleukin-1 cytokine family. This gene and eight other interleukin-1 family genes form a cytokine gene cluster on chromosome 2. The activity of this cytokine is mediated via the interleukin-1 receptor-like 2 (IL1RL2/IL1R-rp2/IL-36 receptor), and is specifically inhibited by interleukin-36 receptor antagonist, (IL-36RA/IL1F5/IL-1 delta). Interferon-gamma, tumor necrosis factor-alpha and interleukin-1β (IL-1β) are reported to stimulate the expression of this cytokine in keratinocytes. The expression of this cytokine in keratinocytes can also be induced by a multiple Pathogen-Associated Molecular Patterns (PAMPs). Both IL-36γ mRNA and protein have been linked to psoriasis lesions and has been used as a biomarker for differentiating between eczema and psoriasis. As with many other interleukin-1 family cytokines IL-36γ requires proteolytic cleavage of its N-terminus for full biological activity. However, unlike IL-1β the activation of IL-36γ is inflammasome-independent and is specifically cleaved by the protease cathepsin S.

As used herein the term “IL-36γ inhibitor” a small molecule, antibody or functional portion or fragment thereof, proteins, peptides, siRNAs, antagonists, agonists, compounds, or nucleotide constructs which either reversibly or irreversibly bind IL-36γ and prevent its binding to a IL-36γ receptor on a cell or tissue in a subject. The term can also mean a small molecule, antibody or functional portion or fragment thereof, proteins, peptides, siRNAs, antagonists, agonists, compounds, or nucleotide constructs which either reversibly or irreversibly bind IL-36γ receptors in an antagonistic manner such that IL-36γ and its analogs or derivatives cannot stimulate the IL-36γ receptors in cells and tissues in a subject. Examples of IL-36γ inhibitors include, but are not limited to, IL-36Ra, and IL-38.

In some embodiments, the IL-36γ inhibitor prevents the production of a biologically active IL-36 by preventing the activation that occurs by proteolytic processing of IL-36, including one or more of IL-36α, IL-36β and/or IL-36γ, to prevent and/or reduce the pro-inflammatory effects of IL-36 including IL-36α, IL-36β and/or IL-36γ. In some embodiments, these inhibitors include peptides of 3 to 10 amino acids in length as described in U.S. Patent Publication No. 2017/0281716, and incorporated by reference herein.

In accordance with an embodiment, the present invention provides a method for reducing or treating the progression of a fibrosis associated disease in a subject in need thereof comprising administering to the subject an effective amount of an IL-17 and/or IL36γ inhibitor.

In accordance with an embodiment, the present invention provides a method for reducing or treating the progression of fibrosis in a subject having an autoimmune disease comprising administering to the subject an effective amount of an IL-17 and/or IL36γ inhibitor.

In accordance with an embodiment, the present invention provides a method for reducing or treating the progression of a fibrosis associated disease or condition in a subject in need thereof, comprising reducing or inhibiting a cell or population of cells expressing the F1 macrophage subtype in the subject by inhibition of the transcription or expression of the IL-18 gene in the cell or population of cells in the subject.

In accordance with an embodiment, the present invention provides a method for reducing or treating the progression of a fibrosis at the site of a surgical procedure in a subject comprising administering to the site of the subject an effective amount of an IL-17 and/or IL36γ inhibitor.

It will be understood by those of ordinary skill in the art, that the treatments described herein of the subject can comprise administration of an IL-17 or IL36γ inhibitor singly, or in combination, or sequentially.

In accordance with some embodiments, the present invention provides methods of treatment, as described above, which further comprise administration of one or more additional biologically active agents. The administration of the additional biologically active agents can be concurrent with, or sequentially to the administration of an IL-17 and/or IL36γ inhibitors to the subject.

In accordance with an embodiment, the present invention provides a method for reducing or treating the progression of a fibrosis associated disease or condition in a subject in need thereof, comprising administering to the subject an effective amount of an IL-18 inhibitor.

In accordance with an embodiment, the present invention provides a method for reducing or treating the progression of a fibrosis associated disease or condition in a subject in need thereof, comprising reducing or inhibiting a cell or population of cells expressing the F2 macrophage subtype in the subject by inhibition of the transcription or expression of one or more of the genes selected from the group consisting of: Il36 gamma, Il17 receptor A, triggering receptor expressed on myeloid cells 1 (Trem-1), aspartic peptidase retroviral like 1 (Asprv1), Toll-like receptor 2 (Tlr2), secretory leukocyte peptidase inhibitor (Slpi), and histidine decarboxylase (Hdc), in the cell or population of cells in the subject.

In accordance with an embodiment, the present invention provides a method for reducing or treating the progression of a fibrosis associated disease or condition in a subject in need thereof, comprising administering to the subject an effective amount of an inhibitor of one or more of the proteins selected from the group consisting of: Il36 gamma, Il17 receptor A, triggering receptor expressed on myeloid cells 1 (Trem-1), aspartic peptidase retroviral like 1 (Asprv1), Toll-like receptor 2 (Tlr2), secretory leukocyte peptidase inhibitor (Slpi), and histidine decarboxylase (Hdc).

As used herein, the term “an inhibitor” means a small molecule, antibody or functional portion or fragment thereof, proteins, peptides, siRNAs, antagonists, agonists, compounds, or nucleotide constructs which either reversibly or irreversibly bind the gene expression product identified herein, and prevent its binding to a receptor on a cell or tissue in a subject. The term can also mean a small molecule, antibody or functional portion or fragment thereof, proteins, peptides, siRNAs, antagonists, agonists, compounds, or nucleotide constructs which either reversibly or irreversibly bind the gene expression products identified herein or their receptors in an antagonistic manner such that the gene expression products identified herein and its analogs or derivatives cannot stimulate the target receptors in cells and tissues in a subject.

In accordance with an embodiment, the present invention provides a method for improving regenerative healing in a tissue injury of a subject comprising increasing a cell or population of cells expressing the R1 and/or R2 macrophage subtype in a subject in need thereof.

In accordance with an embodiment, the present invention provides a method for improving regenerative healing in a wound of a subject in need thereof, comprising increasing a cell or population of cells expressing the R2 macrophage subtype by inhibition of proteins associated with the R1 subtype, including granzyme A (CTLA 3, Gzma), CD52, CAMPATH 1-Antigen (which is inhibited by Alemtuzumab, for example), lipoprotein lipase, CD209, and C—C motif chemokine receptor 2, for example.

In accordance with an embodiment, the present invention provides a method for improving regenerative healing in a wound of a subject in need thereof, comprising any of the methods described herein and further comprising administering to the subject one or more cytokines secreted by a cell or population of cells expressing the R2 macrophage subtype including, for example, C—C motif chemokine ligand 8 (CCl8) C—C motif chemokine ligand 24 (CCl24).

In accordance with an embodiment, the present invention provides a method for improving regenerative healing in a wound of a subject in need thereof, by increasing a cell or population of cells expressing the R1 and/or R2 macrophage subtype comprising increasing the transcription or expression of the monoglyceride lipase (MGL) gene in the cell or population of macrophage cells in a subject.

In accordance with an embodiment, the present invention provides a method for improving regenerative healing in a wound of a subject in need thereof, by increasing a cell or population of cells expressing the R1 and/or R2 macrophage subtype comprising increasing the transcription or expression of the CD301 gene in the cell or population of macrophage cells in a subject.

“Treating” or “treatment” is an art-recognized term which includes curing as well as ameliorating at least one symptom of any condition or disease. Treating includes reducing the likelihood of a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder or condition, e.g., causing any level of regression of the disease; inhibiting the disease, disorder or condition, e.g., impeding its progress; and relieving the disease, disorder or condition, even if the underlying pathophysiology is not affected or other symptoms remain at the same level.

The dose of the inhibitors, such as IL-17 and/or IL36γ and the others, as set forth above, of the present invention also will be determined by the existence, nature and extent of any adverse side effects that might accompany the administration of a particular compound. Typically, an attending physician will decide the dosage of the compound with which to treat each individual patient, taking into consideration a variety of factors, such as age, body weight, general health, diet, sex, compound to be administered, route of administration, and the severity of the condition being treated. By way of example, and not intending to limit the invention, the dose of the compound can be about 0.001 to about 1000 mg/kg body weight of the subject being treated/day, from about 0.01 to about 100 mg/kg body weight/day, about 0.1 mg to about 10 mg/kg body weight/day, including 0.5, 1.0, 2.0, 3.0, 4.0, 5.0, 6.0, 7.0, 8.0, and 9.0 mg/kg body weight/day.

Small Interfering RNA

In particular embodiments, the inhibitors are “small interfering RNA molecules” (“siRNA molecules” or “siRNA”), methods of making siRNA molecules and methods for using siRNA molecules (e.g., research and/or therapeutic methods). The siRNAs of this invention encompass any siRNAs that can modulate the expression of the target genes of interest as described herein.

In a specific embodiment, the siRNA of the present invention may comprise double-stranded small interfering RNA molecules (ds-siRNA). A ds-siRNA molecule of the present invention may be a duplex made up of a sense strand and a complementary antisense strand, the antisense strand being sufficiently complementary to target genes of interest mRNA to mediate RNAi. The siRNA molecule may comprise about 10 to about 50 or more nucleotides. More specifically, the siRNA molecule may comprise about 16 to about 30, e.g., 16, 17, 18, 19, 20, 21, 22, 23, 24, 25, 26, 27, 28, 29, or 30 nucleotides in each strand. The strands may be aligned such that there are at least 1, 2, or 3 bases at the end of the strands which do not align (e.g., for which no complementary bases occur in the opposing strand) such that an overhang of 1, 2 or 3 residues occurs at one or both ends of the duplex when strands are annealed.

In an alternative embodiment, the siRNA of the present invention may comprise single-stranded small interfering RNA molecules (ss-siRNA). Similar to the ds-siRNA molecules, the ss-siRNA molecule may comprise about 10 to about 50 or more nucleotides. More specifically, the ss-siRNA molecule may comprise about 15 to about 45 or more nucleotides. Alternatively, the ss-siRNA molecule may comprise about 19 to about 40 nucleotides. The ss-siRNA molecules of the present invention comprise a sequence that is “sufficiently complementary” to a target mRNA sequence to direct target-specific RNA interference (RNAi), as defined herein, e.g., the ss-siRNA has a sequence sufficient to trigger the destruction of the target mRNA by the RNAi machinery or process. In one embodiment, the ss-siRNA molecule can be designed such that every residue is complementary to a residue in the target molecule. Alternatively, substitutions can be made within the molecule to increase stability and/or enhance processing activity of the molecule. Substitutions can be made within the strand or can be made to residues at the ends of the strand. In a specific embodiment, the 5′-terminus may be phosphorylated (e.g., comprises a phosphate, diphosphate, or triphosphate group). In another embodiment, the 3′ end of an siRNA may be a hydroxyl group in order to facilitate RNAi, as there is no requirement for a 3′ hydroxyl group when the active agent is a ss-siRNA molecule. In other instances, the 3′ end (e.g., C3 of the 3′ sugar) of ss-siRNA molecule may lack a hydroxyl group (e.g., ss-siRNA molecules lacking a 3′ hydroxyl or C3 hydroxyl on the 3′ sugar (e.g., ribose or deoxyribose).

In another aspect, the siRNA inhibitors of the present invention may be modified to improve stability under in vitro and/or in vivo conditions, including, for example, in serum and in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference. For example, the absence of a 2′ hydroxyl may significantly enhance the nuclease resistance of the siRNAs in tissue culture medium.

Furthermore, the siRNA inhibitors of the present invention may include modifications to the sugar-phosphate backbone or nucleosides. These modifications can be tailored to promote selective genetic inhibition, while avoiding a general panic response reported to be generated by siRNA in some cells. In addition, modifications can be introduced in the bases to protect siRNAs from the action of one or more endogenous enzymes.

In an embodiment of the present invention, the siRNA inhibitor may contain at least one modified nucleotide analogue. The nucleotide analogues may be located at positions where the target-specific activity, e.g., the RNAi mediating activity is not substantially effected, e.g., in a region at the 5′-end and/or the 3′-end of the RNA molecule. Particularly, the ends may be stabilized by incorporating modified nucleotide analogues. Examples of nucleotide analogues include sugar- and/or backbone-modified ribonucleotides (e.g., include modifications to the phosphate-sugar backbone). For example, the phosphodiester linkages of natural RNA may be modified to include at least one of a nitrogen or sulfur heteroatom. In backbone-modified ribonucleotides, the phosphoroester group connecting to adjacent ribonucleotides may be replaced by a modified group, e.g., a phosphorothioate group. In sugar-modified ribonucleotides, the 2′ OH-group may be replaced by a group selected from H, OR, R, halo, SH, SR, NH₂, NHR, NR₂ or ON, wherein R is C1-C6 alkyl, alkenyl or alkynyl and halo is F, Cl, Br or I.

Nucleobase-modified ribonucleotides may also be utilized, e.g., ribonucleotides containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; de-aza nucleotides, e.g., 7-deaza-adenosine; O- and N-alkylated nucleotides, e.g., N6-methyl adenosine are suitable. It should be noted that the above modifications may be combined.

Derivatives of siRNA inhibitors may also be utilized herein. For example, cross-linking can be employed to alter the pharmacokinetics of the composition, e.g., to increase half-life in the body. Thus, the present invention includes siRNA derivatives that include siRNA having two complementary strands of nucleic acid, such that the two strands are crosslinked. The present invention also includes siRNA derivatives having a non-nucleic acid moiety conjugated to its 3′ terminus (e.g., a peptide), organic compositions (e.g., a dye), or the like. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The siRNA inhibitors of the present invention can be enzymatically produced or totally or partially synthesized. Moreover, the siRNAs can be synthesized in vivo or in vitro. For siRNAs that are biologically synthesized, an endogenous or a cloned exogenous RNA polymerase may be used for transcription in vivo, and a cloned RNA polymerase can be used in vitro. siRNAs that are chemically or enzymatically synthesized are preferably purified prior to the introduction into the cell.

Although one hundred percent (100%) sequence identity between the siRNA and the target region is preferred in particular embodiments, it is not required to practice the invention. siRNA molecules that contain some degree of modification in the sequence can also be adequately used for the purpose of this invention. Such modifications may include, but are not limited to, mutations, deletions or insertions, whether spontaneously occurring or intentionally introduced.

Moreover, not all positions of a siRNA contribute equally to target recognition. In certain embodiments, for example, mismatches in the center of the siRNA may be critical and could essentially abolish target RNA cleavage. In other embodiments, the 3′ nucleotides of the siRNA do not contribute significantly to specificity of the target recognition. In particular, residues 3′ of the siRNA sequence which is complementary to the target RNA (e.g., the guide sequence) may not critical for target RNA cleavage.

Sequence identity may be determined by sequence comparison and alignment algorithms known to those of ordinary skill in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (e.g., % homology=# of identical positions/total # of positions ×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (e.g., a local alignment). A non-limiting example of a local alignment algorithm utilized for the comparison of sequences is the algorithm of Karlin and Altschul, 87 PNAS USA 2264-68 (1990), and as modified as in Karlin and Altschul 90 PNAS USA 5873-77 (1993). Such an algorithm is incorporated into the BLAST programs (version 2.0) of Altschul, et al., 215 J Mol. Bio 403-10 (1990).

In another embodiment, the alignment may be optimized by introducing appropriate gaps and determining percent identity over the length of the aligned sequences (e.g., a gapped alignment). To obtain gapped alignments for comparison purposes, Gapped BLAST can be utilized as described in Altschul et al., 25(17) Nucl. Acids Res. 3389-3402 (1997). In another embodiment, the alignment may be optimized by introducing appropriate gaps and determining percent identity over the entire length of the sequences aligned (e.g., a global alignment). A non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

In particular embodiments, greater than 90% sequence identity, e.g., 91%, 92%, 93%, 94%, 95%, 96%, 97%, 98%, 99% or even 100% sequence identity, between the siRNA and the portion of the target gene may be used. Alternatively, the siRNA may be defined functionally as a nucleotide sequence (or oligonucleotide sequence) that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). Additional hybridization conditions include, but are not limited to, hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length can be about 5-10° C. less than the melting temperature (Tm) of the hybrid, where Tm is determined according to the following equations. For hybrids less than 18 base pairs in length, Tm (° C.)=2 (# of A+T bases)+4 (# of G+C bases). For hybrids between 18 and 49 base pairs in length, Tm (° C.)=81.5+16.6 (log 10[Na⁺])+0.41 (% G+C)−(600/N), where N is the number of bases in the hybrid, and [Na] is the concentration of sodium ions in the hybridization buffer ([Na⁺] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 50 or more bases.

Other Compositions for Targeting Genes of Interest DNA or mRNA

Antisense molecules can act in various stages of transcription, splicing and translation to block the expression of a target gene. Without being limited by theory, antisense molecules can inhibit the expression of a target gene by inhibiting transcription initiation by forming a triple strand, inhibiting transcription initiation by forming a hybrid at an RNA polymerase binding site, impeding transcription by hybridizing with an RNA molecule being synthesized, repressing splicing by hybridizing at the junction of an exon and an intron or at the spliceosome formation site, blocking the translocation of an mRNA from nucleus to cytoplasm by hybridization, repressing translation by hybridizing at the translation initiation factor binding site or ribosome biding site, inhibiting peptide chain elongation by hybridizing with the coding region or polysome binding site of an mRNA, or repressing gene expression by hybridizing at the sites of interaction between nucleic acids and proteins. An example of an antisense oligonucleotide of the present invention is a cDNA that, when introduced into a cell, transcribes into an RNA molecule having a sequence complementary to at least part of the target gene of interest mRNA.

Furthermore, antisense oligonucleotides of the present invention include oligonucleotides having modified sugar-phosphodiester backbones or other sugar linkages, which can provide stability against endonuclease attacks. The present invention also encompasses antisense oligonucleotides that are covalently attached to an organic or other moiety that increase their affinity for a target nucleic acid sequence. For example, intercalating agents, alkylating agents, and metal complexes can be also attached to the antisense oligonucleotides of the present invention to modify their binding specificities.

The present invention also provides ribozymes as a tool to inhibit target gene of interest expression. Ribozymes are enzymatic RNA molecules capable of catalyzing the specific cleavage of RNA. The characteristics of ribozymes are well-known in the art. See, e.g., Rossi, 4 Current Biol. 469-71 (1994). Without being limited by theory, the mechanism of ribozyme action involves sequence specific hybridization of the ribozyme molecule to complementary target RNA, followed by an endonucleolytic cleavage. In particular embodiments, the ribozyme molecules include one or more sequences complementary to the target gene mRNA, and include the well-known catalytic sequence responsible for mRNA cleavage. See U.S. Pat. No. 5,093,246, incorporated by reference herein. Using the known sequence of the target gene mRNA, a restriction enzyme-like ribozyme can be prepared using standard techniques.

The expression of the target genes can also be inhibited by using triple helix formation. Nucleic acid molecules to be used in triple helix formation for the inhibition of transcription can be single stranded and composed of deoxynucleotides. The base composition of these oligonucleotides must be designed to promote triple helix formation via Hoogsteen base paring rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementarity to a purine-rich region of a single strand of the duplex in a parallel orientation to that strand. In addition, nucleic acid molecules that are purine-rich, e.g., containing a stretch of G residues, may be chosen. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′,3′-5′ manner, such that they base pair first with one strand of a duplex and then the other, eliminating the necessity for a sizeable stretch of either purines or pyrimidines to be present on one strand of a duplex.

The expression of target genes on interest may be also inhibited by what is referred to as “co-repression.” Co-repression refers to the phenomenon in which, when a gene having an identical or similar to the target sequence is introduced to a cell, expression of both introduced and endogenous genes becomes repressed. This phenomenon, although first observed in plant system, has been observed in certain animal systems as well. The sequence of the gene to be introduced does not have to be identical to the target sequence, but sufficient homology allows the co-repression to occur. The determination of the extent of homology depends on individual cases, and is within the ordinary skill in the art.

It would be readily apparent to one of ordinary skill in the art that other methods of gene expression inhibition that selectively target a gene of interest DNA or mRNA can also be used in connection with this invention without departing from the spirit of the invention. In a specific embodiment, using techniques known to those of ordinary skill in the art, the present invention contemplates affecting the promoter region of the gene of interest to effectively switch off transcription.

“Prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term, “carrier,” refers to a diluent, adjuvant, excipient or vehicle with which the therapeutic is administered. Such physiological carriers can be sterile liquids, such as water and oils, including those of petroleum, animal, vegetable or synthetic origin, such as peanut oil, soybean oil, mineral oil, sesame oil and the like. Water is a suitable carrier when the pharmaceutical composition is administered intravenously. Saline solutions and aqueous dextrose and glycerol solutions also can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents.

An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

Further examples of biologically active agents include, without limitation, enzymes, receptor antagonists or agonists, hormones, growth factors, antibiotics, antimicrobial agents, and other antibodies.

Non-limiting examples of biologically active agents include following: anabolic agents, androgenic steroids, anti-infective agents, anti-inflammatory agents such as steroids, non-steroidal anti-inflammatory agents, anti-pyretic and analgesic agents, biologicals, diagnostic agents, growth factors, neuromuscular drugs, nutritional substances, peripheral vasodilators, and prodrugs.

Specific examples of useful biologically active agents the above categories include: antipyretics and analgesics such as acetaminophen, aspirin and ibuprofen; biologicals such as peptides, polypeptides, proteins and amino acids, hormones, interferons or cytokines and other bioactive peptidic compounds; anti-infective agents such as antiseptics and antibiotics; musculoskeletal agents, such as anti-gout anti-inflammatory agents, corticosteroid anti-inflammatory agents, gold compound anti-inflammatory agents, immunosuppressive anti-inflammatory agents, nonsteroidal anti-inflammatory drugs, salicylate anti-inflammatory agents, skeletal muscle relaxants, neuromuscular blocker skeletal muscle relaxants, and reverse neuromuscular blocker skeletal muscle relaxants.

Pharmaceutically acceptable salts are art-recognized, and include relatively non-toxic, inorganic and organic acid addition salts of compositions of the present invention, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di-, or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenthylamine; (trihydroxymethyl) aminoethane; and the like, see, for example, J. Pharm. Sci., 66: 1-19 (1977).

Further examples of biologically active agents include, without limitation, enzymes, receptor antagonists or agonists, hormones, growth factors, autogenous bone marrow, antibiotics, antimicrobial agents, and antibodies. The term “biologically active agent” is also intended to encompass various cell types and genes that can be incorporated into the compositions of the invention.

Buffers, acids and bases may be incorporated in the compositions to adjust pH. Agents to increase the diffusion distance of agents released from the composition may also be included.

The charge, lipophilicity or hydrophilicity of a composition may be modified by employing an additive. For example, surfactants may be used to enhance miscibility of poorly miscible liquids. Examples of suitable surfactants include dextran, polysorbates and sodium lauryl sulfate. In general, surfactants are used in low concentrations, generally less than about 5%.

The specific method used to formulate the novel formulations described herein is not critical to the present invention and can be selected from a physiological buffer (Feigner et al., U.S. Pat. No. 5,589,466 (1996)).

Therapeutic formulations of the product may be prepared for storage as lyophilized formulations or aqueous solutions by mixing the product having the desired degree of purity with optional pharmaceutically acceptable carriers, diluents, excipients or stabilizers typically employed in the art, i.e., buffering agents, stabilizing agents, preservatives, isotonifiers, non-ionic detergents, antioxidants and other miscellaneous additives, see Remington's Pharmaceutical Sciences, 16th ed., Osol, ed. (1980). Such additives are generally nontoxic to the recipients at the dosages and concentrations employed, hence, the excipients, diluents, carriers and so on are pharmaceutically acceptable.

The compositions can take the form of solutions, suspensions, emulsions, powders, sustained-release formulations, depots and the like. Examples of suitable carriers are described in “Remington's Pharmaceutical Sciences,” Martin. Such compositions will contain an effective amount of the biopolymer of interest, preferably in purified form, together with a suitable amount of carrier so as to provide the form for proper administration to the patient. As known in the art, the formulation will be constructed to suit the mode of administration.

Buffering agents help to maintain the pH in the range which approximates physiological conditions. Buffers are preferably present at a concentration ranging from about 2 mM to about 50 mM. Suitable buffering agents for use with the instant invention include both organic and inorganic acids, and salts thereof, such as citrate buffers (e.g., monosodium citrate-disodium citrate mixture, citric acid-trisodium citrate mixture, citric acid-monosodium citrate mixture etc.), succinate buffers (e.g., succinic acid monosodium succinate mixture, succinic acid-sodium hydroxide mixture, succinic acid-disodium succinate mixture etc.), tartrate buffers (e.g., tartaric acid-sodium tartrate mixture, tartaric acid-potassium tartrate mixture, tartaric acid-sodium hydroxide mixture etc.), fumarate buffers (e.g., fumaric acid-monosodium fumarate mixture, fumaric acid-disodium fumarate mixture, monosodium fumarate-disodium fumarate mixture etc.), gluconate buffers (e.g., gluconic acid-sodium glyconate mixture, gluconic acid-sodium hydroxide mixture, gluconic acid-potassium gluconate mixture etc.), oxalate buffers (e.g., oxalic acid-sodium oxalate mixture, oxalic acid-sodium hydroxide mixture, oxalic acid-potassium oxalate mixture etc.), lactate buffers (e.g., lactic acid-sodium lactate mixture, lactic acid-sodium hydroxide mixture, lactic acid-potassium lactate mixture etc.) and acetate buffers (e.g., acetic acid-sodium acetate mixture, acetic acid-sodium hydroxide mixture etc.). Phosphate buffers, carbonate buffers, histidine buffers, trimethylamine salts, such as Tris, HEPES and other such known buffers can be used.

Preservatives may be added to retard microbial growth, and may be added in amounts ranging from 0.2%-1% (w/v). Suitable preservatives for use with the present invention include phenol, benzyl alcohol, m-cresol, octadecyldimethylbenzyl ammonium chloride, benzyaconium halides (e.g., chloride, bromide and iodide), hexamethonium chloride, alkyl parabens, such as, methyl or propyl paraben, catechol, resorcinol, cyclohexanol and 3-pentanol.

Isotonicifiers are present to ensure physiological isotonicity of liquid compositions of the instant invention and include polyhydric sugar alcohols, preferably trihydric or higher sugar alcohols, such as glycerin, erythritol, arabitol, xylitol, sorbitol and mannitol. Polyhydric alcohols can be present in an amount of between about 0.1% to about 25%, by weight, preferably 1% to 5% taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols; amino acids, such as arginine, lysine, glycine, glutamine, asparagine, histidine, alanine, ornithine, L-leucine, 2-phenylalanine, glutamic acid, threonine etc.; organic sugars or sugar alcohols, such as lactose, trehalose, stachyose, arabitol, erythritol, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thiosulfate; low molecular weight polypeptides (i.e., <10 residues); proteins, such as human serum albumin, bovine serum albumin, gelatin or immunoglobulins; hydrophilic polymers, such as polyvinylpyrrolidone, saccharides, monosaccharides, such as xylose, mannose, fructose or glucose; disaccharides, such as lactose, maltose and sucrose; trisaccharides, such as raffinose; polysaccharides, such as, dextran and so on. Stabilizers can be present in the range from 0.1 to 10,000 w/w per part of biopolymer.

Additional miscellaneous excipients include bulking agents, (e.g., starch), chelating agents (e.g., EDTA), antioxidants (e.g., ascorbic acid, methionine or vitamin E) and cosolvents.

Non-ionic surfactants or detergents (also known as “wetting agents”) may be added to help solubilize the therapeutic agent, as well as to protect the therapeutic protein against agitation-induced aggregation, which also permits the formulation to be exposed to shear surface stresses without causing denaturation of the protein. Suitable non-ionic surfactants include polysorbates (20, 80 etc.), poloxamers (184, 188 etc.), Pluronic® polyols and polyoxyethylene sorbitan monoethers (TWEEN-20®, TWEEN-80® etc.). Non-ionic surfactants may be present in a range of about 0.05 mg/ml to about 1.0 mg/ml, preferably about 0.07 mg/ml to about 0.2 mg/ml.

The instant invention encompasses formulations, such as, liquid formulations having stability at temperatures found in a commercial refrigerator and freezer found in the office of a physician or laboratory, such as from about 20° C. to about 5° C., said stability assessed, for example, by microscopic analysis, for storage purposes, such as for about 60 days, for about 120 days, for about 180 days, for about a year, for about 2 years or more. The liquid formulations of the present invention also exhibit stability, as assessed, for example, by particle analysis, at room temperatures, for at least a few hours, such as one hour, two hours or about three hours prior to use.

Examples of diluents include a phosphate buffered saline, buffer for buffering against gastric acid in the bladder, such as citrate buffer (pH 7.4) containing sucrose, bicarbonate buffer (pH 7.4) alone, or bicarbonate buffer (pH 7.4) containing ascorbic acid, lactose, or aspartame. Examples of carriers include proteins, e.g., as found in skim milk, sugars, e.g., sucrose, or polyvinylpyrrolidone. Typically, these carriers would be used at a concentration of about 0.1-90% (w/v) but preferably at a range of 1-10%

The formulations to be used for in vivo administration must be sterile. That can be accomplished, for example, by filtration through sterile filtration membranes. For example, the formulations of the present invention may be sterilized by filtration.

In some aspects, the present inhibitors and methods can be formulated in accordance with the teachings of PCT Application Publication No. WO 2017/027353, entitled “Compositions and Methods for Modulating Wound Healing and Regeneration,” which is hereby incorporated by reference herein as if set forth in its entirety.

Generally, the ingredients are supplied either separately or mixed together in unit dosage form, for example, as a dry lyophilized powder or water-free concentrate in a sealed container, such as an ampule or sachet indicating the quantity of active agent. Where the composition is to be administered by infusion, it can be dispensed with an infusion bottle containing sterile pharmaceutical grade water or saline. Where the composition is administered by injection, an ampule of sterile water for injection or saline can be provided, for example, in a kit, so that the ingredients may be mixed prior to administration.

An article of manufacture containing materials useful for the treatment of the disorders described above is provided. The article of manufacture comprises a container and a label. Suitable containers include, for example, bottles, vials, syringes and test tubes. The containers may be formed from a variety of materials such as glass or plastic. The container holds a composition which is effective for preventing or treating, for example, a wound or a joint disease and may have a sterile access port (for example, the container may be a vial having a stopper pierceable by a hypodermic injection needle). The label on or associated with the container indicates that the composition is used for treating the condition of choice. The article of manufacture may further comprise a second container comprising a pharmaceutically acceptable buffer, such as phosphate-buffered saline, Ringer's solution and dextrose solution. It may further include other materials desirable from a commercial and user standpoint, including buffers, diluents, filters, needles, syringes and package inserts with instructions for use.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, .gamma.-carboxyglutamate, and O-phosphoserine.

The term “amino acid analogs,” refers to compounds that have the same basic chemical structure as a naturally occurring amino acid, e.g., an .alpha. carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid “mimetics” refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As to amino acid sequences, one of ordinary skill in the art recognizes that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typical conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The subject referred to in the inventive methods can be any subject. Preferably, the subject is a mammal. As used herein, the term “mammal” refers to any mammal, including, but not limited to, mammals of the order Rodentia, such as mice and hamsters, and mammals of the order Lagomorpha, such as rabbits. It is preferred that the mammals are from the order Camivora, including Felines (cats) and Canines (dogs). It is more preferred that the mammals are from the order Artiodactyla, including Bovine (cows) and Swine (pigs) or of the order Perssodactyla, including Equine (horses). It is most preferred that the mammals are of the order Primates, Ceboids, or Simoids (monkeys) or of the order Anthropoids (humans and apes). An especially preferred mammal is the human.

The polypeptides, proteins, (including functional portions and functional variants thereof), nucleic acids, recombinant expression vectors, host cells (including populations thereof), and antibodies (including antigen binding portions thereof), can be isolated and/or purified. The term “isolated” as used herein means having been removed from its natural environment. The term “purified” as used herein means having been increased in purity, wherein “purity” is a relative term, and not to be necessarily construed as absolute purity. For example, the purity can be at least about 50%, can be greater than 60%, 70% or 80%, or can be 100%.

Coatings for Use with Methods

It will be understood by those of ordinary skill in the art, that the inhibitors of the F1 and F2 macrophage subtype could be applied to a device to be implanted into a subject via a biologically compatible biomaterial coating.

In accordance with an embodiment, the present invention provides a method for reducing or treating the progression of a fibrosis at the site of a surgical implant in a subject comprising coating the implant with an effective amount of an IL-17 and/or IL36γ inhibitor prior to implantation in the subject.

Such coatings with the ability to incorporate the inhibitors and other compounds that enhance wound healing/tissue regeneration (e.g., agents that specifically increase the ability of a biomaterial scaffold to recruit immune system molecules that enhance wound healing). Examples of such ECM collagen scaffolds and coatings can be found in U.S. Pat. No. 8,673,333, entitled “Cross-Linked Polymer Matrices, and Methods of Making and Using Same,” which is incorporated by reference herein as if set forth in its entirety.

It will be understood by those of ordinary skill that these matrices and coatings can be applied to the implanted device prior to, or concurrently with implantation. Examples of such devices include, but are not limited to, breast implants, reconstructive implants associated with reconstructive or cosmetic surgery, arthroscopic implants, such as artificial joints or sockets, ocular implants, spinal implants, and the like.

Significant to a product of interest is the enhanced integration with the surrounding tissue to increase stability and bonding to a biological surface and to formation of new tissue. In vitro studies have proven efficacy of the chemical mechanism of reacting to the surface and the increased mechanical strength of the material-cell/tissue/organ interface.

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### EXAMPLES

Surgical Procedures and Implantation.

All animal procedures were performed in accordance with an approved JHU IACUC protocol. Female mice wild type C57BL/6j (Jackson Laboratories) and transgenic IL17a^{-/-} (Y. Iwakura, University of Tokyo, Tokyo, Japan) and IL17ra^{-/-} (T. Mustelin, Amgen, Seattle) at ages from 8 to 10 weeks, were aged six to ten weeks-old. The muscle defect was created as previously described (Sadtler, Estrellas et al. 2016). The resulting bilateral muscle defects were filled with 20 mg of a synthetic material or biological scaffold material. As a synthetic materials poly caprolactone (PCL) was tested (particulate, Mn=50,000 g/mol, mean particle size <600 μm, Polysciences). Further, biological scaffold materials, decellularized urinary bladder matrix (Matristem, Acell) were implanted from 0.05 ml of a 400 mg/ml suspension. Control surgeries were injected with 0.05 ml of PBS (no implant control). All materials were UV sterilized prior to use. Directly after surgery, mice were given subcutaneous carprofen (Rimadyl®, Zoetis) at 5 mg/kg for pain relief. For sample harvest, mice were euthanized, and at 1, 3, 6-weeks post-surgery. Additionally, modes of implantation were subcutaneous and intra-muscular injection. Briefly, synthetic materials were implanted subcutaneously (30 mg) on both flanks of the mice or injected directly into the quadricep muscles through a 16 G syringe (5 mg of materials).

Specimen Harvest.

Murine samples were obtained by dissecting the quadriceps femoris muscle followed by fine dicing. Tissues were digested for 45 min at 37° C. with 1.67 Wunsch U/ml Liberase TL (Roche Diagnostics) and 0.2 mg/mL DNase I (Roche Diagnostics) in RPMI 1640 medium (Gibco). The digested tissues were ground through 70 μm cell strainers (ThermoFisher Scientific) and rinsed with DPBS+BSA 0.05% bovine serum albumin, and then washed twice with 1×DPBS. The enriched single cell suspension was washed, and stained with the following antibody panels, respective to application.

Flow Cytometry and Fluorescence Activated Cell-Sorting.

For cell isolation using FACS, suspensions of single cells from digested muscles were stained for 20 minutes at 4° C. using Viability Dye eFluor™ 780 (eBioscience). Further, staining in an antibody panel was conducted 30 minutes at 4° C. including F4/80 PE-Cy7 (BioLegend), CD11b AlexaFluor700 (BioLegend), CD64 PerCP-Cy5.5 (BioLegend), MHCII (I-A/I-E) AlexaFluor488 (BioLegend), CD3 APC (BioLegend), Ly6c BrilliantViolet510 (BioLegend), CD45 BrilliantViolet605 (BioLegend) and Fc Block TruStain fcX (anti-mouse CD16/32) (BioLegend). Sorting of murine macrophages (CD45+F4/80^{hi+}CD64+) was performed from live, CD45+CD3− using a BD FACSAria II (BD Biosciences). The post-sort purity of macrophages used for single cell RNA sequencing was >98%. For subset analysis, an Attune NxT Flow Cytometer (ThermoFisher Scientific) after viability stain, a panel comprised of F4/80 PE-Cy7 (BioLegend), CD9 FITC (BioLegend), CD11c AlexaFluor700 (BioLegend), MHCII (I-A/I-E) PE-CF594 (eBiosciences), CD301 PE (BioLegend), CD206 APC (BioLegend), CD86 BrilliantViolet410 (BioLegend), Ly6c BrilliantViolet510 (BioLegend), CD45 BrilliantViolet605 (BioLegend), Fc Block TruStain fcX (anti-mouse CD16/32) (BioLegend). Data analysis was performed in FlowJo Flow Cytometry Analysis Software (Treestar). For tSNE projection of multi-dimensional flowcytometry datasets, down sampling of macrophages (CD45+F4/80^{hi+}) was performed to 50,400 cells per sample of regenerative, fibrotic, control conditions. Then samples were concatenated and tSNE projections was computed (Iterations=1000, Perplexity=30). For visualization of separation macrophage subtypes were back gated onto 2-dimensional projection.

TABLE 1
Macrophage FACS Panel

| Fluorophore | Marker | Manufacturer |
|---|---|---|
| Efluor780 | Live/Dead | Life Technologies |
| AF700 | CD11b | eBioscience |
| PerCP-Cy5.5 | CD64 | BioLegend |
| PE-Cy7 | F4/80 | BioLegend |
| APC | CD3 | BioLegend |
| AF488 | MHCII | BioLegend |
| BV510 | Ly6c | BioLegend |
| BV605 | CD45 | BioLegend |

TABLE 2
Macrophage Subtype Panel

| Fluorophore | Marker | Manufacturer |
|---|---|---|
| Efluor780 | Live/Dead | Life Technologies |
| FITC | CD9 | BioLegend |
| PE | CD301 | BioLegend |
| PE-CF594 | CD11c | eBioscience |
| PE-594 | MHCII | BioLegend |
| PE-Cy7 | F4/80 | BioLegend |
| APC | CD206 | BioLegend |
| AF700 | CD11c | BioLegend |
| BV421 | CD86 | BioLegend |
| BV510 | Ly6c | Biolegend |
| BV605 | CD45 | Biolegend |

Histopathology and Immunofluorescence Microscopy.

After harvest, implanted tissues at 1, 3, 6, and 12 weeks after implantation were fixed in 10% neutral buffered formalin for 48 hours before ethanol and xylene dehydration. Archival formalin-fixed paraffin-embedded tissues from patients with conditions known to involve increased tissue macrophages, such as Langerhans cell histiocytosis, juvenile xanthogranuloma, and dermal scar, were obtained from the Johns Hopkins Hospital surgical pathology archives. Human tissue samples including silicone breast implants were acquired from patients undergoing implant exchange or replacement surgery exemption IRB00088842. The average age was 56 years old (range of 41 to 70 years old), and the average implant residence time was 41 months (range of 1 to 360 months). Paraffin-embedded sections of 5 μm thickness were produced using a microtome (Leica RM2255 microtome). immunofluorescence staining for CD9, IL-36γ, F4/80, and CD301b was performed with a tyramide signal amplification method using consecutive staining with Opal-520, Opal-570, and Opal-650, respectively. Histological slides were rehydrated in an ethanol to water gradient. After post-fixation in 10% neutral buffered formalin for 30 min, antigen retrieval was conducted in 1×AR6 buffer (Perkin-Elmer) at 95° C. for 15 min. After cooling, endogenous peroxidases were quenched in 3% (v/v) aqueous peroxide (Sigma-Aldrich). All staining was conducted at room temperature after blocking with antibody block/diluent (Perkin-Elmer). For each staining round, the primary antibody in block/diluent (Perkin-Elmer) was incubated at room temperature for 60 min, followed by 10 min of incubation with horseradish peroxidase (HRP) polymer-conjugated secondary antibody, and 10 min of Opal reagent (1:150) in 1× plus amplification diluent (Perkin-Elmer). The antibody HRP complex was stripped at 95° C. 1×AR6 buffer (Perkin-Elmer) for 15 min. After quenching of autofluorescence in 0.04% (w/v) Sudan Black (Sigma-Aldrich) dissolved in 70% (v/v) ethanol, slides were then counterstained with 4′,6-diamidino-2-phenylindole (DAPI) for 5 min before being mounted using Dako mounting medium. Imaging of the histological samples was performed on Zeiss Axio Observer with Apotome.2 and Zeiss Zen Blue software version 2.5. Phagocytosis assay.

Single Cell Encapsulation and Library Generation.

After sorting of Macrophages (CD45⁺F4/80^{hi+}), single cells were encapsulated in water-in-oil emulsion along with gel beads coated with unique molecular barcodes using the 10× Genomics Chromium Single-Cell Platform. For single cell RNA library generation, the manufacturers protocol was performed (10× Single Cell 3′ v2). Sequencing was performed using an Illumina HiSeq2500 Rapid Mode with 310 million reads per sample and a sequencing configuration of 26×8×98 (UMI×Index×Transcript read). The Cell Ranger pipeline software was used to align reads and generate expression matrices for downstream analysis.

Phagocytosis Assay

Macrophage subsets R1 and R2 were sorted according to the gating strategy described in previously. Around 100,000 cells were collected in RPMI 1640+5% (v/v) fetal bovine serum (Gibco). Macrophages were seeded at 60,000 cells/cm2 and incubated at 37° C. for 12 hours. Then, phagocytosis was conducted for 4 hours at a concentration of 1.0×10⁶ cells/ml with Fluoresbrite flash red (Polysciences) with an average diameter of 2.00-μm particles at 2.0×10⁸ particles/ml. Last, DAPI and Alexa Fluor 594 anti-phalloidin were used to counter stain macrophages for fluorescent microscopy of bead phagocytosis of fluorescent beads.

Cell Culture

For macrophage and T cell coculture experiments, bone marrow-derived monocytes (BMDMs) were isolated and differentiated into macrophage as previously described. At day 0, BMDMs were seeded at 60,000 cells/cm2 and incubated at 37° C. for 7 days in medium supplemented with macrophage colony-stimulating factor (100 ng/ml) (PeproTech) into a 12-well insert of a transwell plate (Corning). Naïve CD4+ T cells were isolated from murine lymph nodes at day 4 using a magnetic bead negative selection kit (Miltenyi). Then, T cells were seeded at 1 million cells/ml in 2 ml into six-well plates (Corning) in naïve condition supplemented with IL-2 (100 ng/ml) (PeproTech) or skewed toward the T_{H}17 phenotype using a commercially available kit (R&D Systems). At day 7, differentiated macrophages were exposed to naïve and T_{H}17 T cells at 250,000 cells/cm2 or IL-17A, IL-17F (PeproTech, 100 ng/ml), and control medium for 72 hours.

Fibroblasts were isolated using previously reported methods (45), seeded to T-75 tissue culture plates (Corning), and passaged once before reseeding at 50,000 cells/cm2 for 24 hours and exposed to IL-36γ(R&D Systems) or IL-17A (PeproTech) at 100 ng/ml for 24 hours. Cells were lysed using RLT plus buffer (Qiagen) reagent for quantitative reverse transcription polymerase chain reaction (qRT-PCR).

qRT-PCR Gene Expression Assay

For total and enriched mRNA, lysis was conducted on whole tissues using TRIzol reagent or in vitro cell culture using RLT plus buffer containing 1% (v/v) β-mercaptoethanol. RNA purification was performed with RNeasy Plus Micro and Mini kits (Qiagen). All qRT-PCR was performed using TaqMan Gene Expression Master Mix (Applied Biosystems) according to the manufacturer's instructions. Briefly, 2 μg of enriched mRNA was used to synthesize complementary DNA (cDNA) using Superscript IV VILO Master Mix (Thermo Fisher Scientific). The cDNA concentration was set to 50 to 100 ng/well (in a total volume of 20 μl of PCR) to match the manufacturer recommendations. The qRT-PCRs were performed on the StepOne Plus Real-Time PCR System (Thermo Fisher Scientific), as TaqMan single-plex assays, using the manufacturer recommended settings for quantitative and relative expression. For tissue samples, β2m was used as the reference gene and samples were normalized to PBS-treated controls. All qRT-PCR data were analyzed using the Livak method, where ΔΔCₜ values are calculated and reported as relative quantification (RQ) values calculated by 2^{-ΔΔCt} (50). RQ values are represented by the geometric means, with error bars representing the geometric standard deviation. Low-expressing mRNA transcripts were preamplified using the TaqMan Pre-Amp System (Thermo Fisher Scientific) according to the manufacturer recommendations with 14 cycles of amplification with the primer probes of interest.

Single-Cell Encapsulation and Library Generation

After sorting of macrophages (CD45+F4/80hi+), single cells were encapsulated in water-in-oil emulsion along with gel beads coated with unique molecular barcodes using the 10× Genomics Chromium Single-Cell Platform. For single-cell RNA library generation, the manufacturers' protocol was performed (10× Single Cell 3′ v2). Sequencing was performed using an Illumina HiSeq2500 Rapid Mode with 310 million reads per sample and a sequencing configuration of 26×8×98 (UMI×Index×Transcript read). The Cell Ranger pipeline software was used to align reads and generate expression matrices for downstream analysis.

NanoString Gene Expression Analysis and Single-Cell Data Comparison

Macrophage subsets R1, R2 and F1, and F2+FP1 were sorted according to the gating strategy previously. After lysis in RLT plus buffer containing 2-mercaptoethanol, RNA was purified using the RNeasy Plus Micro Kit (Qiagen). The gene expression analysis for n=3 biologically independent samples was conducted with the nCounter Mouse Myeloid Innate Immunity V2 Panel (XT-CSO-MMII2-12, NanoString Technologies). After quantification of RNA using a Qubit RNA fluorometric assay high-sensitivity kit (Thermo Fisher Scientific), 50 ng of RNA per sample was added to a barcoded probe set reagent and hybridized for 18 hours at 65° C. NanoString data were processed using the nSolver 4.0 software kit. The differential expression data were used for a direct comparison with single-cell clusters. All differentially expressed genes (P<0.05 and positive fold change) for each cluster were taken for further analysis. Undetected genes in the single-cell dataset were removed. Differential expression of the sorted clusters was performed on the single-cell dataset using the gene set generated from NanoString. If the NanoString and single-cell differential expression both showed significance (P<0.05) in the same direction for a given gene and cluster, the two techniques were considered in agreement. If there was significance in the opposite direction, the techniques were considered in disagreement. If only one technique found significance, it was noted as “found in one,” and if neither found significance, “not found” was noted for that gene and cluster.

Computational Analysis

Sequence Alignment, Filtering, Normalization, and Scaling.

Alignment was performed with STAR (Dobin, Davis et al. 2013) through the Cell Ranger pipeline. Filtering, normalization, and scaling were performed using Seurat (Villani, Satija et al. 2017, Butler, Hoffman et al. 2018). Cells with UMI counts for fewer than 200 genes and genes with expression in less than 0.1% of cells were both dropped from analysis. Data was normalized by Eₙₒᵣₘ=log(UMI*10,000/UMIₜₒₜₐₗ) where UMIₜₒₜₐₗ is total UMI expression for a given gene. Scaling was performed to remove unwanted effects correlated to batch, total UMI count, percent of mitochondrial genes, and cell cycle. To determine cell cycle scores for use with scaling, Seurat's CellCycleScoring function was used with a previously determined set of genes correlated with G2M or S phase. Data scaling was performed by first fitting a linear model with the parameters to scale out as independent variables (batch, total UMI count, percent mitochondrial genes, G2M score, and S score). For each gene, the residuals from the fit were Z-scored and used for downstream analysis. Finally, principle components were calculated and the top 50 were taken based on leveling of variance per principle component as determined by an elbow plot (not shown).

Clustering Analysis.

SC3 and Seurat's unsupervised clustering algorithms using the top 50 principle components were compared using silhouette coefficients. For each clustering algorithm, a reasonable range of resolution or k parameters were determined a priori. Silhouette coefficients were determined for each result and compared. Seurat was found to have consistently higher silhouette coefficients, and was used. A resolution value was selected based on a combination of high silhouette coefficient and reasonable number of clusters for biological consideration. After clustering, a small population of contaminant fibroblasts were found and removed.

Removal of Low Signal Clusters.

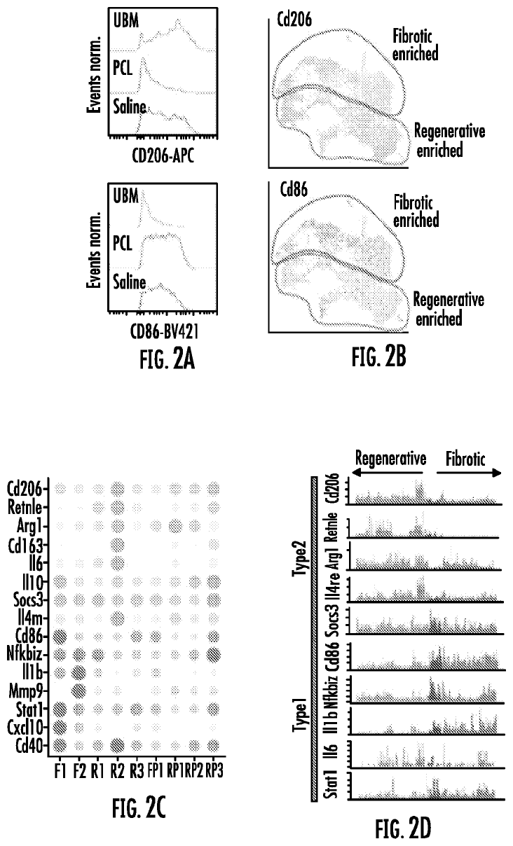

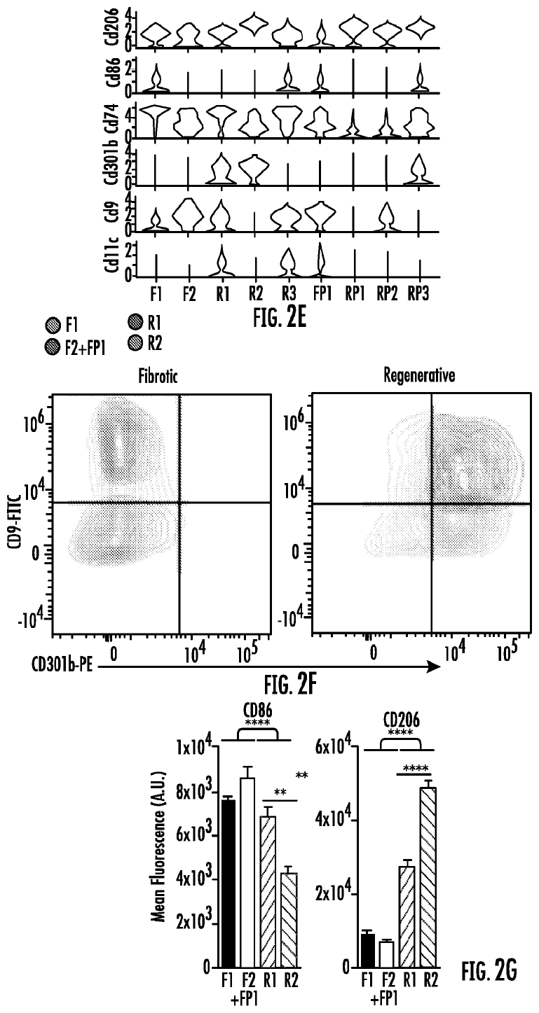

Downstream analysis also found evidence of clusters which had little signal remaining after scaling. To attempt to quantify this, differential expression test was calculated using Seurat's negative binomial statistical test with a minimum log fold-change of 1. Based on these results together with quality control information such as total UMI count (Supplementary FIG. 2), it was decided to remove clusters O1-4 from analysis to avoid diluting the signal from clusters with stronger signal. The resulting data matrix was reprocessed identically to ultimately find 9 clusters of macrophages with strong enough signal to identify surface markers and suggest biological function.

Differential Expression and Gene Set Enrichment.

Differential expression was calculated using edgeR's exactTest (Robinson, McCarthy et al. 2010). Unless otherwise specified, differential expression for a specific cluster was determined by comparison against all other clusters. For heat maps comparing expression of multiple different genes by cluster, gene expression values were normalized against the highest expression value for each given gene. Feature plots and violin plots for gene expression were generated with Seurat's FeaturePlot and VlnPlot respectively, using log-normalized expression values. Ranked gene lists were used to calculate gene set enrichment scores GSEA was performed as previously described using fgsea (Sergushichev 2016). Briefly, all genes in the analysis were ranked by p-value with the exception of the direct comparison between R1 and R2 where genes were ranked by log fold-change. For a given gene set, a running enrichment score is calculated by stepping through the ranked gene list and adding to the score when encountering a gene in the gene set or subtracting from the score otherwise. The enrichment score for a gene set is maximum (or minimum if negative) of the running enrichment score with a higher enrichment score indicating higher concentration of genes in the gene set towards the beginning of the ranked gene list. Finally, a null distribution for the gene set is calculated by repeatedly permuting the ranked genes and used to calculate a p-value. Gene ontology cellular components, biological process, and hallmark gene sets were obtained from the Broad Institute for GSEA.

Protein Network Analysis.

Functional network analysis of gene products in macrophage clusters was conducted using a databank-based query of the STRING consortium (Von Mering, Jensen et al. 2005, Szklarczyk, Franceschini et al. 2014). Three letter codes of the top 300 differentially expressed genes per cluster were mapped to the Mus musculus protein products. Connectivity of proteins in the network was scored ranked from 0 to 1, with 1 being the highest possible confidence weighed meta-data comparison based on experimental protein-protein interaction evidence, Pubmed text mining and curated databases. Gephi network visualization tool was used to plot the cluster protein networks (Bastian, Heymann et al. 2009). Network nodes represent proteins with the size proportional to the number of interactions. Edges represent protein-protein associations with the thickness proportional to the strength of the meta-evidence supporting the interaction.

Pseudotime Analysis.

Pseudotime analysis was performed using Slingshot (Street, Risso et al. 2018). Precursor clusters were determined using differential expression and gene set enrichment for clusters. In particular, it was found that precursor clusters shared gene set enrichment patterns (not shown). R3 was removed as an outlier due to highly unique gene expression. To determine genes associated with pseudotime progression, genes were regressed on pseudotime as determined by Slingshot using a general additive model with the gam R package. The top 10 genes by p-value were then used to generate a heat map, ordering cells by pseudotime. Z-scored scaled residuals expression values were used for the heat maps.

TABLE 3
Materials

| REAGENT or RESOURCE | SOURCE | IDENTIFIER |
|---|---|---|
| PE/Cy7 anti-mouse F4/80 Antibody | Biolegend | Cat # 123114 |
| Alexa Fluor ® 700 anti-mouse/human | Biolegend | Cat # 101222 |
| CD11b Antibody | | |
| Alexa Fluor ® 700 anti-mouse CD11c | Biolegend | Cat # 117320 |
| Antibody | | |
| Ly6C BV510 anti-mouse | Biolegend | Cat # 128033 |
| Brilliant Violet 605(TM) anti-mouse | Biolegend | Cat # 103140 |
| CD45 | | |
| PE anti-mouse CD301 (MGL1/MGL2) | Biolegend | Cat # 145704 |
| Brilliant Violet 421(TM) anti-mouse | Biolegend | Cat # 105031 |
| CD86 | | |
| FITC anti-mouse MHCII | Biolegend | Cat # 107606 |
| Purified anti-mouse F4/80 Antibody | Biolegend | Cat # 123101 |
| CD64 | Biolegend | Cat # 139308 |
| IL1F9 Monoclonal Antibody | Thermo Fisher | Cat # MA5-23907 |
| | Scientific | |
| | Fisher | Cat # 65-0865-14 |
| Liberase(TM) TL Research Grade low | Roche | Cat # 5401020001 |
| Thermolysin concentration | | |
| Micromatrix | Acell | |
| RPMI 1640 | ThermoFisher | Cat # 11875119 |
| | Scientific | |
| Data files for single-cell RNA sequencing | This paper | EGA/GSE # |
| (raw data) | | |
| Data files for bulk RNA sequencing (raw | This paper | EGA/GSE # |
| data) | | |
| Data files for single-cell RNA sequencing | This paper | EGA/GSE # |
| (processed data) | | |
| Oligonucleotides | | |
| Primers (if needed) | This paper (or | N/A |
| TATAGACTAGATGA or w/ever | other paper) | |
| Software and Algorithms | | |
| Cell Ranger | 10x Genomics | 10xgenomics.com |
| Seurat | Butler 2018 | github.com/satijalab/seurat |
| | (Use citation | |
| | software later) | |
| SC3 | Kiselev 2017 | github.com/hemberg-lab/SC3 |
| edgeR | Robinson 2010 | bioconductor.org/packages/ |
| | | release/bioc/html/edgeR.html |
| R-GSEA | Broad Institute | software.broadinstitute.org/ |
| | | gsea/downloads.jsp |
| Network analysis stuff | | |
| Slingshot | Street 2018 | github.com/kstreet13/ |
| | | slingshot |
| Monocle2 | Trapnell 2014, | bioconductor.org/packages/ |
| | Qiu 2017, | release/bioc/html/monocle.html |
| | Qiu 2017 | |
| Gephi | Bastian M., | gephi.org/ |
| | Heymann S., | |
| | Jacomy M. (2009). | |
| STRING version 10 | Szklarczyk et al. | version10.string-db.org |
| | Nucleic Acids Res. | |
| | 2015 43(Database | |
| | issue): D447-52 | |
| gam | Wood 2011 | cran.r-project.org/web/ |
| | | packages/gam/index.html |

### Example 1

Single Cell Profiling of F480hi Macrophages in Regenerative Versus Fibrotic Tissue Environments

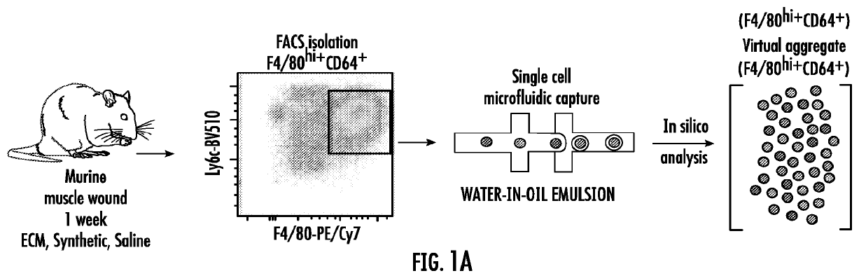

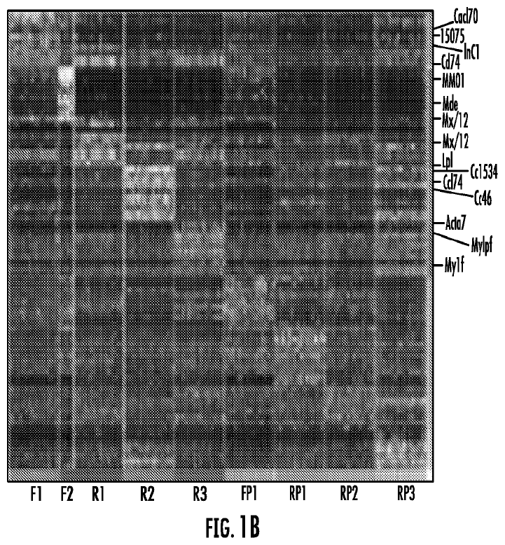

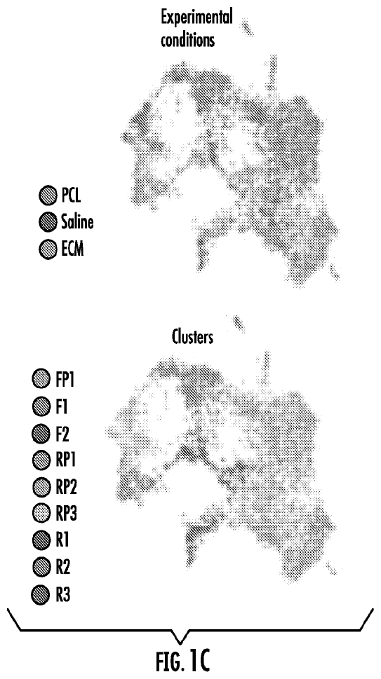

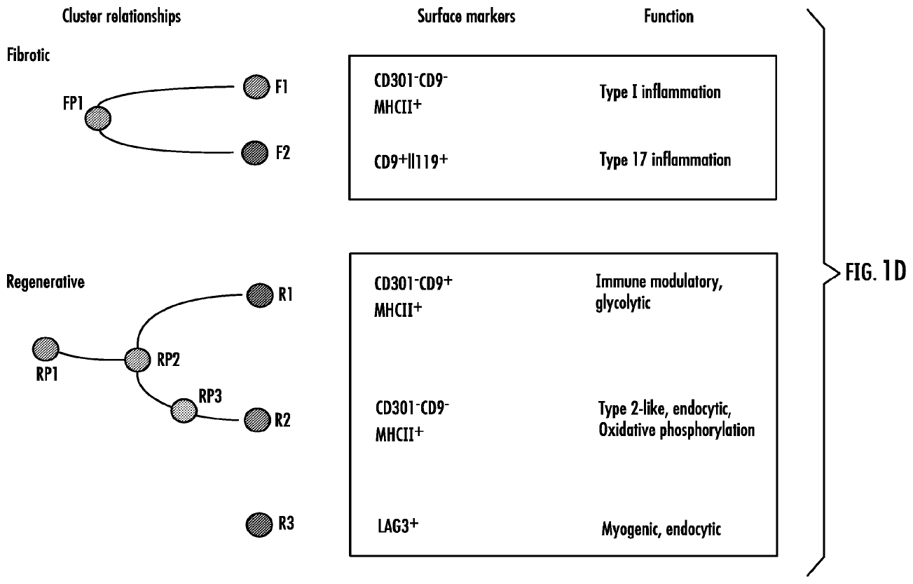

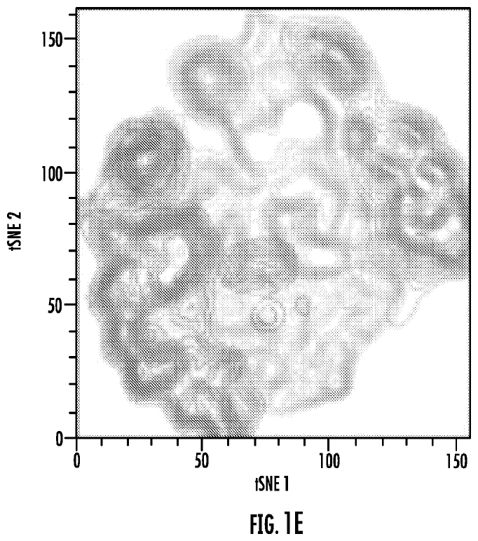

To model tissue microenvironments with activated and heterogeneous macrophage populations, we selected biomaterials that are used clinically and induce divergent immune and tissue responses. Biological scaffolds derived from the extracellular matrix of tissues promote healing of muscle (20) and induce a Type 2 (M2/T_{H}2) immune microenvironment (4). We selected a urinary bladder matrix (UBM), with properties similar to other ECM-derived materials, that is used clinically in wound healing (21) and hernia repair (22). Poly caprolactone (PCL) was selected as a model synthetic biomaterial that stimulates a fibrotic response (23) and a Type 17 (M1/T_{H}17) immune microenvironment (19). When characterizing macrophages in these biomaterial tissue microenvironments, CD86 (a ligand for CD28 and CTLA4) and CD206 (a scavenger receptor) expression is analyzed on differentiated macrophages (CD45⁺CD64⁺F4/80^{hi+}) (24) to characterize polarization. We sorted this population of differentiated macrophages for single cell analysis one week after biomaterial implantation and applied them to the 10× single cell RNA sequencing platform (FIG. 1A). This resulted in the capture of ˜7,200 macrophages with an average read depth of ˜50,000 reads per cell across ˜13,000 genes with over 4,000 median unique molecular identifiers (UMI) counts per cell. By condition, the total number of macrophages captured was 3,343 from UBM, 2,919 from PCL, and 876 from saline.

### Example 2

Unbiased Clustering and Pseudo-Time Trajectories Reveal Functional Diversity that Correlates with Biomaterial Tissue Environment.

Unbiased clustering algorithms categorized macrophages into clusters based on global gene expression patterns. We first computationally pooled macrophages from regenerative (UBM), fibrotic (PCL), and control (saline) conditions to create a virtual aggregate. Cells with reduced signal after scaling were removed from the analysis, leaving 9 computationally determined clusters. The clusters were largely enriched for regenerative or fibrotic macrophages with differential expression analysis confirming distinct gene expression profiles (FIG. 1B). UMAP (uniform manifold approximation and projection), a dimensional reduction algorithm, grouped cells by cluster, indicating that UMAP and the clustering algorithm agreed on the similarity of cell phenotypes (FIG. 1C).

The experimental origin of each cell was then superimposed on the UMAP plot so that the enrichment of cells from regenerative or fibrotic microenvironment could be identified across all cell clusters (FIG. 1C). Cell clusters were distinguished by experimental conditions, so we termed macrophages from the UBM tissue microenvironment regenerative associated clusters (RACs) and those from the PCL tissue microenvironment fibrosis associated clusters (FACs). Potential batch effects were removed by scaling on each experimental condition. Groups of macrophages isolated from saline-treated wounds were found at the interfaces between fibrotic and regenerative macrophages. This intermediate profile is supported by flow cytometry data suggesting that macrophages in a muscle wound without a biomaterial have characteristics of fibrotic and regenerative microenvironment (FIG. 2A). Similar cell numbers were sequenced from the fibrotic and regenerative conditions, but two-thirds of the macrophage cell clusters were RACs suggesting there is increased complexity of macrophage phenotypes in the regenerative tissue microenvironment.

To identify relationships between cell clusters and differentiation trajectories, we performed Slingshot pseudotime and RNA velocity analysis on the RACs and FACs. Precursor clusters (RP1, RP2, FP1) were selected based on similarities in gene expression in clusters across experimental conditions. While RNA velocity, which predicts cell movement on a ˜32 h timescale, confirmed movement of cells from RP2 towards R1 and supported the defined clusters. Pseudotime results indicate a branching lineage in both the RACs (R1, R2) and FACs (F1, F2) (FIG. 1D) with two functionally specialized terminal clusters in each condition. R3 was excluded from the pseudotime analysis due to its unique gene expression profile that included muscle-related genes.

To enable identification of the terminal regenerative and fibrotic macrophages, we determined surface marker combinations in silico that could differentiate subsets experimentally (FIG. 1D). We performed flow cytometry on cells isolated from the UBM, PCL, and saline treatment conditions using the computationally-identified cluster surface markers. The CD45+Ly6c-F4/80^{hi} cell populations from all conditions were concatenated together to create a t-Distributed Stochastic Neighbor Embedding (tSNE) plot containing a complex mixture of all macrophages. We then identified macrophages expressing the surface markers CD9 (a protein involved in cell adhesion, fusion, and motility), CD301b (a galactose C-type lectin), and MHCII in the aggregated data set to represent the in silico macrophage clusters. The four terminal clusters F1, F2 (and FP1), R1, and R2 could be separated in the aggregate, suggesting that the new populations can be readily identified experimentally using flow cytometry (FIG. 1E).

### Example 3

Expression of Canonical Polarization Markers CD86 and CD206 is Distributed Across Macrophage Clusters

The correlation of the unbiased single cell clusters was first explored with canonical M1 and M2 polarization markers. Flow cytometry analysis of macrophages confirmed the enrichment of CD206 in the regenerative condition and CD86 in the synthetic condition with saline or untreated wound exhibiting intermediate levels of both markers (FIG. 2A). Histograms were consistent with previous studies that found UBM treatment downregulates CD86 while CD206 remains constant and PCL slightly decreases CD86 and significantly decreases CD206 compared to saline treatment.

While scRNAseq supported the enrichment of Cd206 across regenerative macrophages and Cd86 across fibrotic macrophages, it could not discriminate between phenotypically distinct macrophages subsets. Expression levels of these two surface markers superimposed on the UMAP plot show neither Cd86 nor Cd206 correlated with the computationally determined clusters (FIG. 2B). We then compared the expression patterns of canonical polarization genes and Cd206 and Cd86 across the unbiased clusters of RACs and FACs and found similar disparities (FIG. 2C). In particular, cluster F1 had elevated expression of Cd86, but relatively low levels of other M1 genes Il1b, Mmp9, and Nfkbiz. Meanwhile, Il1b and Mmp9 were expressed predominantly in fibrotic cluster F2 which had the lowest expression levels of Cd86. The strongest expression of M2 markers was found together with the highest expression of Cd206 in R2, but R2 also had high expression of the M1 marker Cd40. Other clusters had elevated expression of the type 2 associated genes Arg1 and Socs3 despite reduced expression of Cd206.

Comparison of macrophage polarization markers on a per cell basis in the different experimental conditions also revealed a significant heterogeneity (FIG. 2D). Retnla was the only type 2 gene not expressed in the fibrotic macrophages. Expression of other type 2 genes, Arg1 and Socs3, did not parallel Cd206 expression on a cell-by-cell basis. At the same time, high levels of Retnla, Arg1, and Socs3 expression were found in cells that did not express Cd206. There was a similar pattern of disparity in expression of Cd86 and type 1 genes. Cd86 expression did not correlate with Nfkbiz, and Il1b on a cell-by-cell basis. Many cells expressed high levels of Cd86 expression in parallel with low levels of Nfkbiz and Il1b.

### Example 4

CD9, CD301b, and MHCII Expression Identifies Fibrotic and Regenerative Macrophage Subsets.

Since the single cell analysis confirmed that Cd86 and Cd206 expression did not differentiate phenotypic subsets, we explored alternative surface markers in the scRNAseq dataset. In silico assessment of surface markers revealed that differential expression of Cd301b, Cd9, and Cd74 (encoding the MHCII-associated invariant chain) was sufficient to identify each of the macrophage clusters (FIG. 2E). We then tested these surface markers on bulk cell isolates from the tissue environments to confirm that the gene expression correlated with surface protein expression and could separate the macrophage populations using multi-parametric flow cytometry. The proposed surface markers were able to discriminate the macrophage subsets corresponding to in silico determined clusters in the regenerative and fibrotic conditions (FIG. 2F). The surface marker CD301b allowed nearly complete separation of macrophages unique to the regenerative microenvironment while CD9 and CD74 further differentiated macrophages into the multiple in silico predicted subsets (FIG. 2G). The CD9 and CD301b surface marker paradigm differentiates macrophage groups not equivalent to the commonly used cytokine-induced in vitro macrophage phenotypes (IL-4, IFNγ+LPS, IL-10) that all express high levels of CD9.

### Example 5

Computational Phenotyping Reveals Limited Inflammatory and Phagocytic Macrophages in the Regenerative Environment.

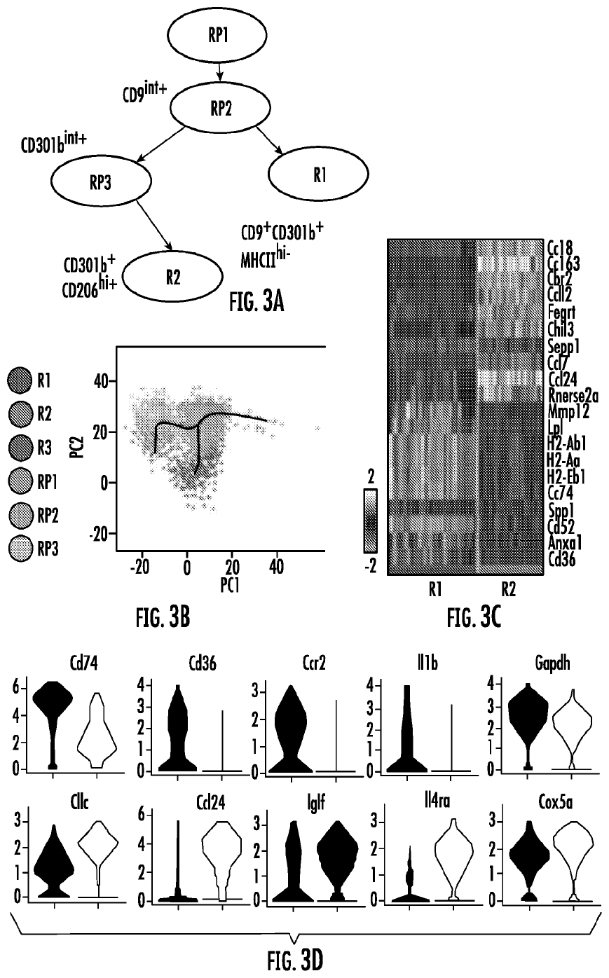

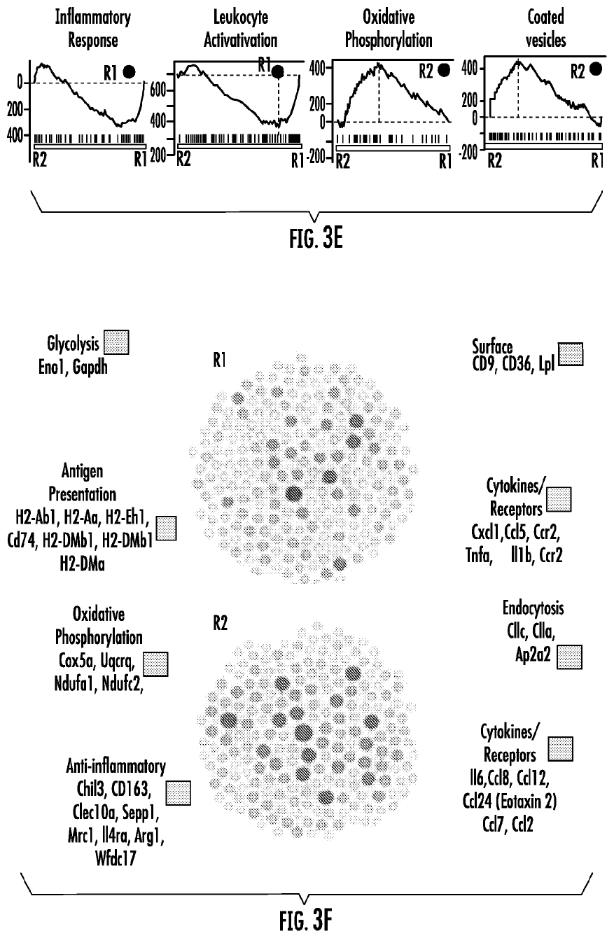

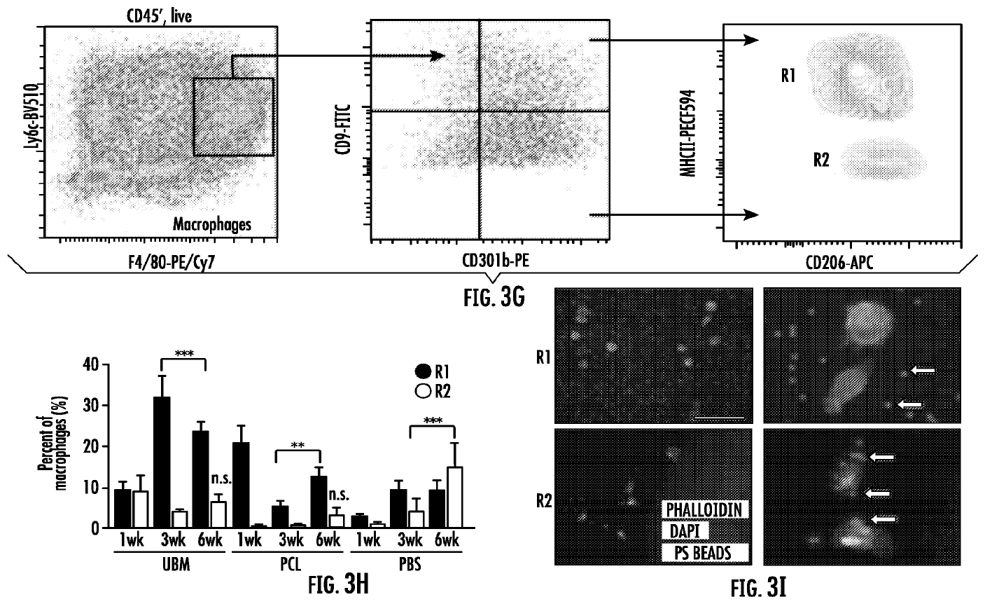

Pseudotime analysis was used to elucidate relationships between the RACs and found a branching differentiation trajectory with two terminally differentiated clusters (R1, R2) stemming from three precursor clusters (RP1-3) (FIG. 3A, B). Precursor RP3, a direct precursor to only R2, shared a similar but reduced phenotype to R2 based on differential gene expression (data not shown). While R2 is composed entirely of macrophages derived from the regenerative condition, RP3 had a relatively high composition of saline derived macrophages (data not shown). The RACs expressed Il4ra with R2 expressing the highest levels, correlating with the UBM induction of IL-4 and the macrophage response to the tissue environment induced by the biological scaffold.

To characterize the phenotype and potential functional properties of R1 and R2, we compared outcomes of their differential expression, gene set enrichment analysis, and gene network analysis. Differential gene expression analysis of R1 showed upregulation of antigen-presenting capacity, inflammatory activity, and glycolysis, including Cd74, Ccr2, Il1b, and Gapdh (FIG. 3C, D). While R1 expression levels of the inflammatory genes Il1b and Tnfa were elevated when compared to other RACs, they were low when compared to the highly inflammatory FACs. Gene set enrichment of R1 also found elevation of inflammatory responses (FIG. 3E). R1 was enriched in leukocyte activation gene sets, suggesting that these macrophages play a role in communication and activation of the adaptive immune system. Finally, network analysis supported both the differential expression and gene set enrichment. R1 expressed gene modules associated with glycolysis (Enol, Gapdh), antigen presentation (H2 genes and Cd74), and inflammatory cytokines and chemokines (Cxcl1, Ccr2, Ccl5, Tnfa, and Il1b) (FIG. 3F).

In contrast to the inflammatory R1 macrophage subset, R2 expressed multiple genes associated with alternatively-activated or anti-inflammatory macrophages (FIG. 3C). Differential expression and network analysis both revealed enrichment for anti-inflammatory genes such as Chil3, Cd163, and Mrc1 (gene encoding CD206) (FIG. 3D). In addition, R2 expressed high levels of Ccl24 (Eotaxin-2), a chemokine for eosinophil attraction that is observed responding to ECM materials (25). Gene set enrichment and gene modules from network analysis also support a unique metabolic profile with expression of Cox5a, Uqcrq, Ndufa1, and Ndufc2 (FIG. 3E, F). This profile supports R2 activation of oxidative phosphorylation compared to glycolysis in R1. R2 expression also included gene set enrichment and endocytic gene modules (Cltc, Clta, and Ap2a2) that suggest phagocytic activity in this subset.

### Example 6

In Vivo Subtyping Validates in Silico Predicted RAC Phenotypes and Microenvironment Enrichment Using CD9 and CD301b-Based Flow Cytometry.

In silico defined clusters R1 and R2 were validated in vivo by flow cytometry using a combination of CD301b, CD9 and MHCII on cells isolated from the UBM tissue microenvironment. While CD301b separated the terminal regenerative clusters R1 and R2 from progenitor clusters and R3, we found the R1 and R2 could be further defined as CD9⁺MHCII⁺ and CD9⁻MHCII⁻ respectively. A combination of both markers provided distinct separation of R1 from R2 for analysis and cell sorting (FIG. 3G). As predicted computationally, CD301b⁺CD9⁺R1 and CD301b⁺CD9⁻R2 macrophages express different levels of both MHCII and CD11c when quantified by surface marker expression using flow cytometry. R1 and R2 are distinguished by higher CD11c and lower CD206 expression respectively. These pronounced subset specific profiles elucidate earlier suggestions of complex phenotypes associated with the regenerative UBM microenvironment (25, 26).

To evaluate the kinetic evolution of the regenerative macrophage subsets in different experimental conditions, we performed flow cytometry on macrophages isolated from regenerative, fibrotic, and control VML microenvironments at 1, 3 and 6 weeks (FIG. 3H). Consistent with RNA velocity analysis (data not shown), R1 increased significantly in the regenerative condition from 1 to 3 weeks while the R2 population maintained elevated levels with respect to the other conditions. As expected, the fibrotic condition had low expression of both R1 and R2 at all time points. The saline wound control showed an increase in both R1 and R2 at 3 weeks and R2 at 6 weeks, suggesting the UBM tissue microenvironment may be following an accelerated course of regeneration that is observed functionally with respect to an untreated wound.

Expression analysis predicted that R2 macrophages were phagocytic, an important function in wound clean up that is associated with tissue repair. Since the surface markers uniquely identified R2 by flow cytometry, we were able to sort pure populations of R1 and R2 and experimentally validate phagocytic activity. R1 and R2 macrophages sorted from a UBM-treated regenerative environment were evaluated for the ability to uptake fluorescent microbeads. The R2 (CD301b*CD9-) macrophage subset phagocytosed beads whereas R1 (CD301b*CD9+) macrophages did not internalize any beads (FIG. 3l). This result confirms the functional properties predicted by the gene expression analysis and provides surface markers that can specifically identify phagocytic macrophages.

### Example 7

Genes Associated with the Local Tissue Environment are Found in Macrophage Cluster R3.

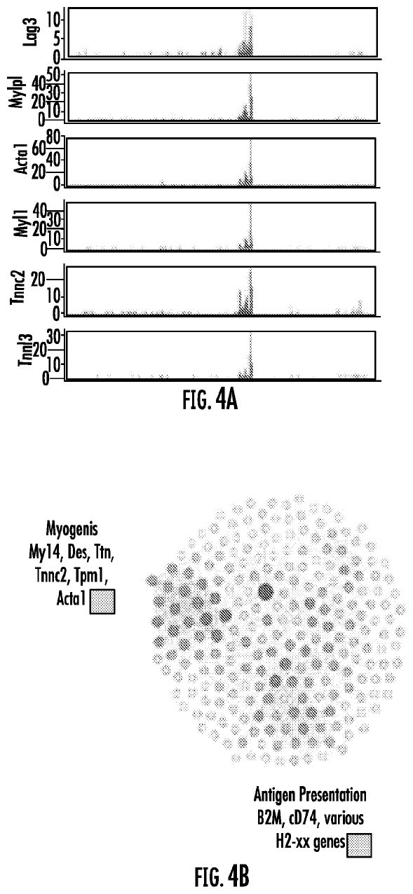

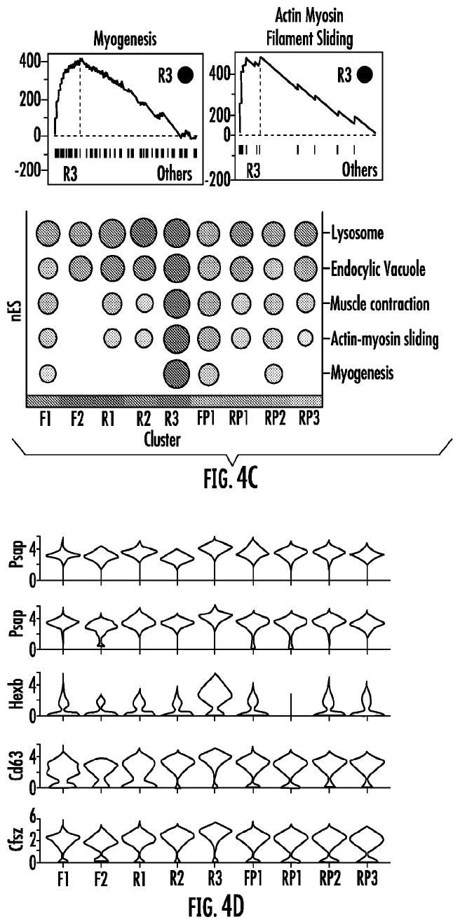

The R3 RAC expressed the most unique gene signature compared to all other subsets with UBM treatment. Many of the top differentially expressed genes in this macrophage population were related to muscle tissue Mylpf Myl1, Acta1, Tnnc2, and Tnnt3 (FIG. 4A) and dendritic cells (Lag3, Cd11c) (27). Gene set enrichment and network analysis indeed supported this finding, with gene modules associated with skeletal muscle (Myl4, Des, Ttn, Tnnc2, Tpm1, and Acta1) (FIG. 4B) and strong enrichment of gene sets related to myogenesis and muscle function (FIG. 4C). Deeper analysis found that R3 cells also expressed genes characteristic of endocytosis and lysosome activity including moderate elevation of Psap, Ctss, Hexb, Cd63, and Ctsz (FIG. 4D). This is further supported by gene set enrichment finding enrichment of sets related to lysosomal and endocytic function and network analysis showing a module of genes associated with antigen presentation (B2m, Cd74, and H2 genes). Two possible explanations emerge from these results. R3 muscle signature may be due to macrophages differentiating towards a muscle lineage and participating in building new muscle tissue since this is the tissue environment in which they are located. Alternatively, the single cell analysis is detecting endosomal mRNA in macrophages that have phagocytosed muscle cells during cell and tissue debris removal in the wound healing process.

### Example 8

Computational Phenotyping Indicates Inflammatory and Autoimmune FAC Subsets.

We also applied pseudotime and RNA velocity analysis to determine FAC differentiation and polarization relationships (data not shown). From pseudotime analysis, one precursor cluster (FP1) led to a branching trajectory with two terminal fibrosis-associated macrophage clusters, F1 and F2 (FIG. 5A). RNA velocity confirmed pseudotime results and continued polarization of F2 from the bulk macrophages. The F1 cluster expressed traditional markers of inflammation and genes associated with the interferon response including Irf7, 1118, and Tlr2 (FIG. 5B). Gene sets for both IFNα and IFNγ response were enriched in F1 (FIG. 5C). Gene networks also showed modules associated with interferon response (Stat1, Myd88, Irf7, and Tlr2) and cytokines associated with inflammatory function (Il18, Ccl4, Ccl7, and Cxcl10) (FIG. 5D). While both macrophage populations have elements of inflammation, F1 and F2 exhibit significantly different expression profiles.

While F2 was small, it had a distinct gene expression signature that included recently discovered cytokines and genes with limited characterization. While F2 expressed inflammatory markers Slpi, Hdc, Tlr2, and Il1b (not shown), it also expressed genes associated with autoimmunity Il36γ, Trem1, Asprv1, and Il17ra (FIG. 5B). The unique nature of this subset and possible functional relevance is exemplified in the expression of Il36γ (also known as Il1f9) that is found in lesions of skin psoriasis and participates in a positive feedback loop in type 17 immune responses (28). IL-17, the primary cytokine of Type 17 responses is also associated with fibrotic diseases not yet associated with autoimmunity including idiopathic pulmonary fibrosis (IPF) (29, 30), cardiac fibrosis (31), and the foreign body response suggesting a Type 17-autoimmune connection. The F2 cluster expressed increased Il17ra, further supporting the role of IL-17 in this subset and the macrophage responsiveness in the PCL tissue microenvironment

We validated experimentally the computationally-defined F1 and F2 surface markers using flow cytometry and immunostaining. F1 and F2 macrophages were isolated using CD301b, CD9, and MHCII. Fibrotic-associated macrophages were first defined as CD301b⁻ macrophages (the marker for RACs). Then F1 was isolated as CD9-MHCII⁺ and F2 as CD9⁺MHCII⁻ (FIG. 5E). CD11c expression matched in silico predicted levels to further differentiate the subsets (FIG. 5F). Immunofluorescent staining confirmed the presence of CD9⁺/F4/80⁺ macrophages near the synthetic material in a 1 week after implantation (FIG. 5F). The F4/80⁺ cells that were in direct contact with synthetic material were CD9⁺. CD9⁺ macrophages were also present in other regions of F4/80⁺ cells in the tissue space between PCL granules resulting in clusters that were CD9⁺ and CD9⁻.

To validate the experimentally the FAC clusters, we performed flow cytometry using the computationally defined surface marker strategy including CD9, CD301b, and MHCII (FIGS. 5E and F). According to the computational analysis, the surface markers differentiate the F1 cluster but the F2 and FP1 clusters are not separated. Both F1 and F2+FP1 were elevated in the PCL fibrotic condition at 1 week with a significant reduction in F1 from 1 to 3 weeks. However, levels of F2+FP1 were consistently elevated, suggesting that F2 may be important in the development of pathological fibrosis. The UBM regenerative condition had low levels of both F1 and F2 at 1 week which was reduced at 3 and 6 weeks. The control wound condition had high levels of F1 at 1 weeks and 3 weeks with a significant decrease at 3 weeks. There was a low, decreasing number of F2 macrophages in the control wound environment, further suggesting that F2 is associated with fibrotic pathology. Finally, we performed immunofluorescence (IF) for F4/80⁺CD9⁺ on tissue treated with PCL to visualize F2 (and FP1) macrophages (FIG. 5G).

### Example 9

The Pathological F2 Macrophage is Dependent on IL-17 and is Present in Human Disease.

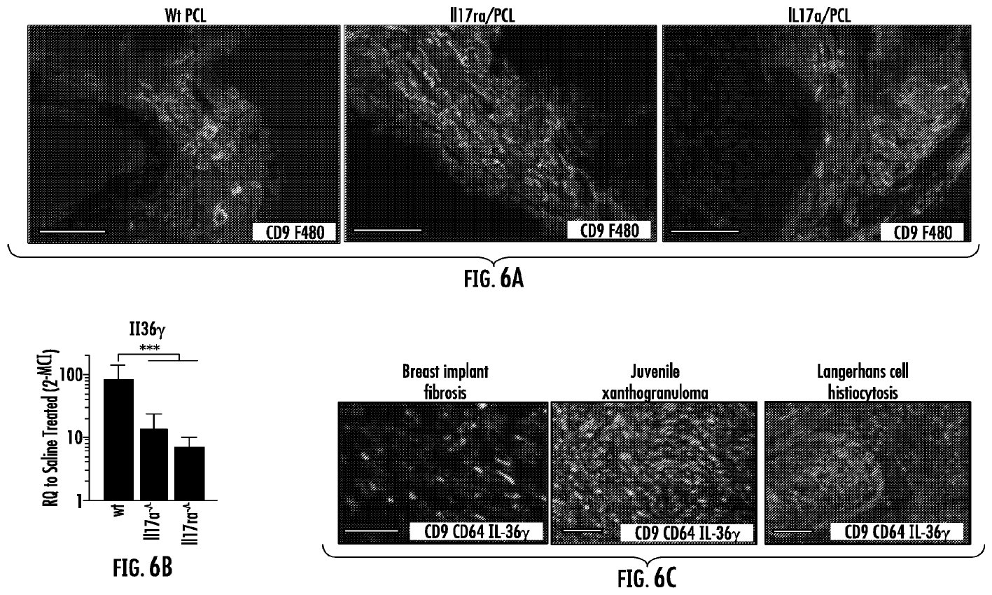

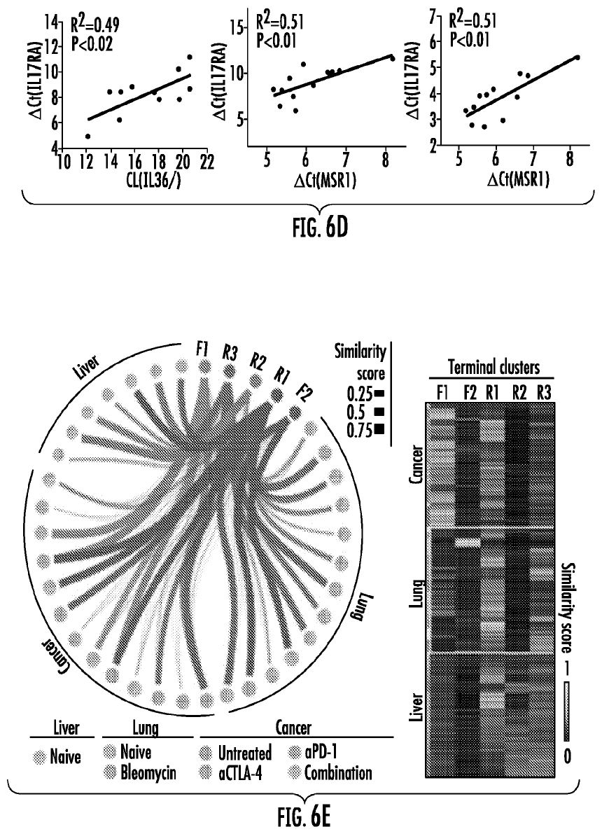

Based on the evidence suggesting F2 is unique to the PCL condition and pathological fibrosis, coupled with the suggested feedback loop between IL-36γ and IL-17, we further evaluated the F2 population and its dependence on IL-17 signaling. PCL was implanted in mice lacking IL-17A (Il17a^{-/-}) or the IL-17 receptor (Il17ra^{-/-}) to ablate IL-17 signaling. Fibrosis in response to PCL decreased in Il17a^{-/-} mice but was only completely abrogated in Il17ra^{-/-} mice (19). Connecting this functional outcome with macrophage responses, immunostaining for CD9 and F/480 decreased significantly in Il17a^{-/-} and Il17ra^{-/-} mice treated with PCL compared to WT after 12 weeks (FIG. 6A). Il36γ coding for a key cytokine expressed in F2 that links IL-17 and autoimmune responses in the tissue, significantly increased after PCL implantation compared to saline in WT animals. Expression of Il36γ decreased significantly in Il17a^{-/-} and Il17ra^{-/-} mice, further supporting a connection between IL-17 signaling and IL-36γ in F2 (FIG. 6B).

The broader relevance of the fibrosis-associated F2 profile was explored in human fibrotic pathologies. Positive immunofluorescence staining for F2-specific markers (CD64⁺CD9⁺IL-36γ⁺) in human breast implant tissue capsules as well as histiocytosis (juvenile xanthogranuloma and Langerhans cell histiocytosis) suggest that these macrophages are also relevant to human disease (FIG. 6C). Further, IL17RA expression is correlated to IL36γ and the expression of the macrophage marker MSR1, supporting a role of the IL-17/IL-36γ feedback loop in human macrophages (FIG. 6D).

### Example 10

Terminal Macrophages Profiles are Present in Human and Murine Tissues.

To determine the broader relevance of the model biomaterial-induced macrophages, we utilized the SingleCellNet program (32) and trained the scRNAseq cell classification algorithm using the terminal macrophage data sets. We then compared the results to macrophages computationally extracted from publicly available data sets of healthy human liver (33), a mouse model of idiopathic pulmonary fibrosis (34), and a mouse model of sarcoma with cancer immunotherapy (15). After clustering macrophages within each data set, we applied SingleCellNet to quantify similarity with the terminal biomaterial macrophages (FIG. 6E). The R1 and R3 macrophage subsets were found in all of the conditions that we evaluated. While the expression of muscle markers in R3 initially suggest that they may be unique to the muscle tissue environment, their presence in all the conditions studied suggest that even this subset has broader relevance in multiple tissue types and pathologies. We found that only liver, a strongly regenerative tissue, contained macrophages similar to R2. The majority of macrophages in the sarcoma were similar to F1 but there was also a small cluster similar to F2. The F2 cluster was also present in the lung tissue.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1. S. Aras, M. R. Zaidi, TAMeless traitors: macrophages in cancer progression and metastasis. British journal of cancer 117, 1583 (2017).
2. V. Kumar, S. Patel, E. Tcyganov, D. I. Gabrilovich, The nature of myeloid-derived suppressor cells in the tumor microenvironment. Trends in immunology 37, 208-220 (2016).
3. S. F. Badylak, J. E. Valentin, A. K. Ravindra, G. P. McCabe, A. M. Stewart-Akers, Macrophage phenotype as a determinant of biologic scaffold remodeling. Tissue Engineering Part A 14, 1835-1842 (2008).
4. K. Sadtler et al., Developing a pro-regenerative biomaterial scaffold microenvironment requires T helper 2 cells. Science 352, 366-370 (2016).
5. T. A. Wynn, K. M. Vannella, Macrophages in tissue repair, regeneration, and fibrosis. Immunity 44, 450-462 (2016).
6. Q. N. Myrvik, E. S. Leake, B. Fariss, Studies on pulmonary alveolar macrophages from the normal rabbit: a technique to procure them in a high state of purity. The Journal of Immunology 86, 128-132 (1961).
7. D. L. Knook, N. Blansjaar, E. C. Sleyster, Isolation and characterization of Kupffer and endothelial cells from the rat liver. Experimental cell research 109, 317-329 (1977).
8. G. J. Guillemin, B. J. Brew, Microglia, macrophages, perivascular macrophages, and pericytes: a review of function and identification. Journal of leukocyte biology 75, 388-397 (2004).
9. Y Lavin et al., Tissue-resident macrophage enhancer landscapes are shaped by the local microenvironment. Cell 159, 1312-1326 (2014).
10. P. J. Murray, T. A. Wynn, Protective and pathogenic functions of macrophage subsets. Nature reviews immunology 11, 723 (2011).
11. A. Mantovani et al., The chemokine system in diverse forms of macrophage activation and polarization. Trends in immunology 25, 677-686 (2004).
12. C. D. Mills, K. Kincaid, J. M. Alt, M. J. Heilman, A. M. Hill, M-1/M-2 macrophages and the Th1/Th2 paradigm. The Journal of Immunology 164, 6166-6173 (2000).
13. D. M. Mosser, J. P. Edwards, Exploring the full spectrum of macrophage activation. Nature reviews immunology 8, 958 (2008).
14. P. J. Murray et al., Macrophage activation and polarization: nomenclature and experimental guidelines. Immunity 41, 14-20 (2014).
15. M. M. Gubin et al., High-dimensional analysis delineates myeloid and lymphoid compartment remodeling during successful immune-checkpoint cancer therapy. Cell 175, 1014-1030 (2018).
16. A. C. Villani et al., Single-cell RNA-seq reveals new types of human blood dendritic cells, monocytes, and progenitors. Science 356, eaah4573 (2017).
17. J. M. Anderson, A. Rodriguez, D. T. Chang. Foreign body reaction to biomaterials, in Seminars in immunology, (Elsevier), vol. 20, pp. 86-100.
18. B. D. Ratner, A. S. Hoffman, F. J. Schoen, J. E. Lemons, Biomaterials science: an introduction to materials in medicine. (Elsevier, 2004).
19. L. Chung et al., Interleukin-17 and senescence regulate the foreign body response. bioRxiv, 583757 (2019).
20. B. M. Sicari et al., An acellular biologic scaffold promotes skeletal muscle formation in mice and humans with volumetric muscle loss. Science translational medicine 6, 234ra258-234ra258 (2014).
21. H. Kimmel, M. Rahn, T. W. Gilbert, The clinical effectiveness in wound healing with extracellular matrix derived from porcine urinary bladder matrix: a case series on severe chronic wounds. The Journal of the American College of Certified Wound Specialists 2, 55-59 (2010).
22. J. Zografakis et al., Urinary bladder matrix reinforcement for laparoscopic hiatal hernia repair. JSLS: Journal of the Society of Laparoendoscopic Surgeons 22, (2018).
23. J. C. Doloff et al., Colony stimulating factor-1 receptor is a central component of the foreign body response to biomaterial implants in rodents and non-human primates. Nature materials 16, 671 (2017).
24. E. L. Gautier et al., Gene-expression profiles and transcriptional regulatory pathways that underlie the identity and diversity of mouse tissue macrophages. Nature immunology 13, 1118 (2012).
25. M. T. Wolf et al., A biologic scaffold-associated type 2 immune microenvironment inhibits tumor formation and synergizes with checkpoint immunotherapy. Science translational medicine 11, eaat7973 (2019).
26. K. Sadtler et al., Divergent immune responses to synthetic and biological scaffolds. Biomaterials 192, 405-415 (2019).
27. C. J. Workman et al., LAG-3 regulates plasmacytoid dendritic cell homeostasis. The Journal of Immunology 182, 1885-1891 (2009).
28. Y. Carrier et al., Inter-regulation of Th17 cytokines and the IL-36 cytokines in vitro and in vivo: implications in psoriasis pathogenesis. Journal of Investigative Dermatology 131, 2428-2437 (2011).
29. L. J. Celada et al., PD-1 up-regulation on CD4+ T cells promotes pulmonary fibrosis through STAT3-mediated IL-17A and TGF-β1 production. Science translational medicine 10, eaar8356 (2018).
30. M. S. Wilson et al., Bleomycin and IL-1β-mediated pulmonary fibrosis is IL-17A dependent. Journal of Experimental Medicine 207, 535-552 (2010).
31. L. Wu et al., Cardiac fibroblasts mediate IL-17A-driven inflammatory dilated cardiomyopathy. Journal of Experimental Medicine 211, 1449-1464 (2014).
32. Y. Tan, P. Cahan, SingleCellNet: a computational tool to classify single cell RNA-Seq data across platforms and across species. bioRxiv, 508085 (2018).
33. S. A. MacParland et al., Single cell RNA sequencing of human liver reveals distinct intrahepatic macrophage populations. Nature communications 9, 4383 (2018).
34. D. Aran et al., Reference-based analysis of lung single-cell sequencing reveals a transitional profibrotic macrophage. Nature immunology, 1 (2019).
35. C. Bleriot et al., Liver-resident macrophage necroptosis orchestrates type 1 microbicidal inflammation and type-2-mediated tissue repair during bacterial infection. Immunity 42, 145-158 (2015).
36. M. F. Pustjens et al., IL4-10 synerkine induces direct and indirect structural cartilage repair in osteoarthritis. Osteoarthritis and Cartilage 24, S532 (2016).
37. K. Sadtler et al., The scaffold immune microenvironment: biomaterial-mediated immune polarization in traumatic and nontraumatic applications. Tissue Engineering Part A 23, 1044-1053 (2017).
38. J. E. Heredia et al., Type 2 innate signals stimulate fibro/adipogenic progenitors to facilitate muscle regeneration. Cell 153, 376-388 (2013).
39. J. C. Rodriguez-Prados et al., Substrate fate in activated macrophages: a comparison between innate, classic, and alternative activation. The Journal of Immunology 185, 605-614 (2010).
40. J. I. Odegaard, A. Chawla, Alternative macrophage activation and metabolism. Annual Review of Pathology: Mechanisms of Disease 6, 275-297 (2011).
41. F. Wang et al., Glycolytic stimulation is not a requirement for M2 macrophage differentiation. Cell metabolism 28, 463-475 (2018).
42. G. Fiorino, P. D. Omodei, Psoriasis and Inflammatory Bowel Disease: Two Sides of the Same Coin? Journal of Crohn's and Colitis 9, 697-698 (2015).
43. S. Kagami, H. L. Rizzo, J. J. Lee, Y. Koguchi, A. Blauvelt, Circulating Th17, Th22, and Th1 cells are increased in psoriasis. Journal of Investigative Dermatology 130, 1373-1383 (2010).
44. J. F. Zambrano-Zaragoza, E. J. Romo-Martinez, M. Durán-Avelar, N. Garcia-Magallanes, N. Vibanco-Pérez, Th17 cells in autoimmune and infectious diseases. International journal of inflammation 2014, (2014).
45. M. T. Mizwicki et al., Tocilizumab attenuates inflammation in ALS patients through inhibition of IL6 receptor signaling. American journal of neurodegenerative disease 1, 305 (2012).
46. X. Wang et al., IL-36γ transforms the tumor microenvironment and promotes type 1 lymphocyte-mediated antitumor immune responses. Cancer cell 28, 296-306 (2015).
47. W. Seo et al., Exosome-mediated activation of toll-like receptor 3 in stellate cells stimulates interleukin-17 production by γδ T cells in liver fibrosis. Hepatology 64, 616-631 (2016).
48. J. W. Ford, D. W. McVicar, TREM and TREM-like receptors in inflammation and disease. Current opinion in immunology 21, 38-46 (2009).
49. M. Schenk, A. Bouchon, F. Seibold, C. Mueller, TREM-1-expressing intestinal macrophages crucially amplify chronic inflammation in experimental colitis and inflammatory bowel diseases. The Journal of clinical investigation 117, 3097-3106 (2007).
50. K. J. Livak, T. D. Schmittgen, Analysis of relative gene expression data using real-time quantitative PCR and the 2−ΔΔCT method. methods 25, 402-408 (2001).
51. A. Dobin et al., STAR: ultrafast universal RNA-seq aligner. Bioinformatics 29, 15-21 (2013).
52. A. Butler, P. Hoffman, P. Smibert, E. Papalexi, R. Satija, Integrating single-cell transcriptomic data across different conditions, technologies, and species. Nature biotechnology 36, 411 (2018).
53. M. S. Kowalczyk et al., Single-cell RNA-seq reveals changes in cell cycle and differentiation programs upon aging of hematopoietic stem cells. Genome research 25, 1860-1872 (2015).
54. V. Y. Kiselev et al., SC3: consensus clustering of single-cell RNA-seq data. Nature methods 14, 483 (2017).
55. M. D. Robinson, D. J. McCarthy, G. K. Smyth, edgeR: a Bioconductor package for differential expression analysis of digital gene expression data. Bioinformatics 26, 139-140 (2010).
56. A. Sergushichev, An algorithm for fast preranked gene set enrichment analysis using cumulative statistic calculation. BioRxiv, 060012 (2016).
57. D. Szklarczyk et al., STRING v10: protein-protein interaction networks, integrated over the tree of life. Nucleic acids research 43, D447-D452 (2014).
58. C. Von Mering et al., STRING: known and predicted protein-protein associations, integrated and transferred across organisms. Nucleic acids research 33, D433-D437 (2005).
59. M. Bastian, S. Heymann, M. Jacomy, Gephi: an open source software for exploring and manipulating networks, in Third International AAAI Conference. (2009).
60. K. Street et al., Slingshot: Cell lineage and pseudotime inference for single-cell transcriptomics. BMC genomics 19, 477 (2018).
61. G. La Manno et al., RNA velocity of single cells. Nature 560, 494 (2018).
62. C. Tabula Muris, Single-cell transcriptomics of 20 mouse organs creates a Tabula Muris. Nature 562, 367 (2018).

1. Use of an effective amount of an IL-17 and/or IL36γ inhibitor for reducing or inhibiting a cell or population of cells expressing the F1 and or F2 macrophage subtype in a subject in need thereof.

2. Use of an effective amount of an IL-17 and/or IL36γ inhibitor for reducing or treating the progression of a fibrosis associated disease in a subject in need thereof.

3. Use of an effective amount of an IL-18 inhibitor for reducing or treating the progression of a fibrosis associated disease or condition in a subject in need thereof.

4. Use of the inhibition of the transcription or expression of one or more of the genes selected from the group consisting of: Il36 gamma, Il17 receptor A, triggering receptor expressed on myeloid cells 1 (Trem-1), aspartic peptidase retroviral like 1 (Asprv1), Toll-like receptor 2 (Tlr2), secretory leukocyte peptidase inhibitor (Slpi), and histidine decarboxylase (Hdc), transmembrane protein 1, lipocalin 2, leucine rich alpha-2-glycoprotein 1, and C—X—C motif chemokine receptor 2, in the cell or population of cells in the subject for reducing or treating the progression of a fibrosis associated disease or condition in a subject in need thereof.

5. The use of any of claims 1-7, wherein the subject is suffering from an autoimmune disease.

6. The use of any of claims 1-7, wherein the subject is suffering from cirrhosis of the liver.

7. The use of any of claims 1-7, wherein the subject is having a surgical procedure.

8. The use of claim 7, wherein the surgical procedure is implantation of a medical device or apparatus.

9. The use of claim 8, wherein the use further comprises coating the implant with an effective amount of one or more antagonists, or inhibitors of gene expression selected from the group consisting of: an IL-17 antagonist; an IL36γ antagonist; IL-18 antagonist; Il36 gamma inhibitor; Il17 receptor A inhibitor; triggering receptor expressed on myeloid cells 1 (Trem-1) inhibitor; aspartic peptidase retroviral like 1 (Asprv1) inhibitor; Toll-like receptor 2 (Tlr2) inhibitor; secretory leukocyte peptidase inhibitor (Slpi) inhibitor; and a histidine decarboxylase (Hdc) inhibitor.

10. Use of the inhibition of proteins associated with the R1 subtype, including granzyme A (CTLA 3, Gzma); CD52; CAMPATH 1-Antigen; lipoprotein lipase; CD209; and C—C motif chemokine receptor 2 for improving regenerative healing in a wound of a subject in need thereof, thereby increasing a cell or population of cells expressing the R2 macrophage subtype.

11. The use of claim 10 wherein the CAMPATH 1-Antigen inhibitor is Alemtuzumab.

12. The use of claim 10, wherein the use further comprises the addition of one or more cytokines secreted by a cell or population of cells expressing the R2 macrophage subtype including C—C motif chemokine ligand 8 (CCl8) and C—C motif chemokine ligand 24 (CCl24).

13. A method for identification of regenerative associated macrophages in a heterogeneous cellular sample, the method comprising:
contacting the heterogeneous cellular sample comprising regenerative associated macrophages, fibrotic associated macrophages, and other macrophages with a CD301 specific binding member; and
distinguishing the regenerative associated macrophages from the other macrophages as those macrophages where the CD301 cell surface marker specific binding member has bound to the macrophage.

14. A method for identification of a subpopulation of regenerative associated macrophages known as R1 macrophages in a heterogeneous cellular sample, the method comprising:
contacting a sample of regenerative associated macrophages that were previously distinguished with a CD301 specific binding member binding to the macrophages and exposing the macrophages with a CD9 and MHCII specific binding member; and
distinguishing the R1 macrophages based on whether the CD9 and MHCII surface marker specific binding members both bind to the cell surface markers on macrophages of the sample.

15. A method for identification of a subpopulation of regenerative associated macrophages known as R2 macrophages in a heterogeneous cellular sample, the method comprising:
contacting a sample of regenerative associated macrophages that were previously distinguished with a CD301 specific binding member binding to the macrophages and exposing the macrophages with a CD9 and MHCII specific binding member; and
distinguishing the R2 macrophages from the other macrophages in the sample based on whether neither of the CD9 and MHCII surface marker specific binding members bind to the cell surface markers on macrophages of the sample.

16. A method for identification of fibrotic associated macrophages in a heterogeneous cellular sample, the method comprising:
contacting the heterogeneous cellular sample comprising regenerative associated macrophages, fibrotic associated macrophages, and other macrophages with a CD301 specific binding member; and
distinguishing the fibrotic associated macrophages from the other macrophages in the sample based on whether the CD301 cell surface marker specific binding member does not bind to a cell surface marker on macrophages of the sample.

17. A method for identification of a subpopulation of fibrotic associated macrophages known as F1 macrophages in a heterogeneous cellular sample, the method comprising:
contacting a sample of the fibrotic associated macrophages that were previously distinguished with a CD301 specific binding member and which did not bind to the macrophages with a CD9 and MHCII specific binding member; and
distinguishing the F1 macrophages from the other macrophages in the sample based on whether the CD9 surface marker specific binding member does not bind to the cell surface markers and the MHCII specific binding member does bind to the cell surface markers on macrophages of the sample.

18. A method for identification of a subpopulation of fibrotic associated macrophages known as F2 macrophages in a heterogeneous cellular sample, the method comprising:
contacting a sample of the fibrotic associated macrophages that were previously distinguished with a CD301 specific binding member which did not bind to the macrophages with a CD9 and MHCII specific binding member; and
distinguishing the F2 macrophages from the other macrophages in the sample based on whether the CD9 surface marker specific binding member binds to the cell surface markers and the MHCII specific binding member does not bind to the cell surface markers on macrophages of the sample.

19. The method according to any of claims 13 to 18, wherein distinguishing comprises at least one of separating, enriching, identifying and providing an assessment.

20. The method according to any of claims 13 to 19, wherein one or more of the specific binding members comprises an antibody or a fragment thereof.

21. The method according to any of claims 13 to 20, wherein the distinguishing comprises flow cytometry.
